(19) United States
(12) Patent Application Publication
(10) Pub. No.: US 2019/0015233 A1
(43) Pub. Date: Jan. 17, 2019

(54) Fabric-Based Soft Actuators

(71) Applicant: PRESIDENT AND FELLOWS OF HARVARD COLLEGE, Cambridge, MA (US)

(72) Inventors: Kevin C. GALLOWAY, Nashville, TN (US); Diana WAGNER, Somerville, MA (US); Conor WALSH, Cambridge, MA (US); Siddharth SANAN, Sunnyvale, CA (US); Rachael GRANBERRY, St. Paul, MN (US)

(73) Assignee: President and Fellows of Harvard College, Cambridge, MA (US)

(21) Appl. No.: 16/068,290

(22) PCT Filed: Jan. 5, 2017
(86) PCT No.: PCT/US2017/012303

§ 371 Date: Jul. 5, 2018

Related U.S. Application Data
(60) Provisional application No. 62/275,015, filed on Jan. 5, 2016.

(51) Int. Cl. A61F 5/01; F15B 15/10; A61H 9/00; A61B 17/135; A61F 2/58; A61F 5/058

(52) U.S. Cl. CPC A61F 5/013; F15B 15/10; A61H 9/0078; A61B 17/135; A61F 2002/5012; A61F 5/012; A61F 5/05816; A61H 2201/165; A61F 2/586

(57) ABSTRACT

A fabric-based soft actuator includes a first fabric layer, a second (material) layer, a bladder, and a fluid pump. The first fabric layer has anisotropic or isotropic stretch properties. The second layer is a fabric layer with anisotropic or isotropic stretch properties and/or a strain-limiting layer. The bladder is disposed between or integrated with the first fabric layer and the second layer, while the fluid pump is in fluid communication with and configured to inflate the bladder.

### GOVERNMENT SUPPORT

This invention was made with government support under Grant No. NSF IIS-1317744 awarded by the National Science Foundation. The Government has certain rights in the invention.

### BACKGROUND

Soft fluidic actuators have seen significant interest in recent years as an alternative to traditional electro-magnetic actuation technologies. Compared to traditional actuators, such as electromagnetic or rigid hydraulic actuators, soft fluidic actuators offer potential advantages in terms of weight, compliance and fabrication cost. Additionally, soft fluidic actuators can be mechanically programmed to generate complex motions using only a single input, such as pressurized gas or liquid, as described in PCT Application Publication No. WO 2015/066143 A1, PCT Application Publication No. WO 2015/050852 A1, and PCT Application Publication No. WO 2015/102723 A2.

Perhaps the most widely applied example of a soft fluidic actuator is the McKibben actuator. McKibben actuators exhibit linear contraction in response to pressure changes. McKibben actuators essentially consist of a balloon or bladder that is placed inside a braided shell. The braided shell functions to constrain the expansion of the balloon and results in the characteristic motion of the actuator. An ideal McKibben actuator has zero strain energy associated with its motion. Since the functional elements of the actuator only support tensile loads, the overall structure of the actuator can be extremely lightweight.

Soft actuators for prescribing other types of motions, such as bending and twisting, have relied largely on the use of elastomers and fibers to achieve the desired motion. FIG. 1 presents examples of previous designs for actuators 10 where strain-limiting layers 12 and fiber reinforcements 14 can be applied to an elastomeric (rubber) body 16 to control the deformation of the rubber body 16 under fluid pressurization and to generate a variety of output motions, including bending (A), bend-twisting (B), extending (C), and extend-twisting (D). Compared to McKibben actuators, these actuators tend to be relatively heavy and less efficient (i.e., require higher operating pressures) because of the work needed to strain the elastomer during actuator motion. On the other hand, the mechanics of the braided shell found on the McKibben actuator is extremely limiting when more complex motions are desired.

### SUMMARY

A fabric-based soft actuator and methods for its fabrication and use are described herein, where various embodiments of the apparatus and methods may include some or all of the elements, features and steps described below.

As described herein, a fabric-based soft actuator includes a first fabric layer, a second (material) layer, a bladder, and a pressure source (e.g., a fluid pump). The first fabric layer has anisotropic or isotropic stretch properties. The second layer is a fabric layer with anisotropic or isotropic stretch properties and/or a strain-limiting layer. The bladder is disposed between or integrated with the first fabric layer and the second layer, while the pressure source is in fluid communication with and configured to inflate the bladder.

The fabric-based soft actuators can be lightweight and efficient, while being able to generate complex motions. Fabric-based soft actuators, as disclosed herein, can be manufactured by sewing or bonding two or more material layers together to define a pocket and by positioning a bladder or fabric coating configured to hold pressurized fluid inside the pocket. The resulting fabric-based actuator may then be actuated by adding a pressurized fluid to the bladder. The use of fabrics allows a lightweight construction owing to the relatively low thickness of the fabrics (usually less than 1 mm) while, at the same time, offering significant strength in tension. A common fabric material, such as inextensible twisted thread, is usually less than 0.5 mm in thickness, and its failure load limit can be greater than 1000 N/m.

In some embodiments, one of the material layers is made of a knit material that can be sewn together with other layers to define the geometry of the actuator. These constructions can also be achieved with chemical and thermal bonds or a combination thereof. In other embodiments, the fabric is unitary with a changing knit structure across the fabric. Methods for making and using fabric-based soft actuators are also disclosed herein.

In one aspect, a fabric-based soft actuator is described, including: (1) a fabric sleeve comprising a knit material with anisotropic stretch properties and a strain-limiting layer and (2) a bladder for holding pressure that is separate from and disposed between the material layers that form the fabric sleeve.

The soft actuator described herein can provide a broad range of motions (e.g., bending, extending, twisting, and combinations thereof) and can be very pliable and flexible when uninflated/depressurized. Meanwhile, the actuator, when pressurized, can be very stiff due to the tension on the fabric containing the inflated bladder. Furthermore, the soft actuator can be operated to perform with less input pressure than was needed for previous fiber-reinforced elastomeric soft-actuators, as less fluidic pressure may be needed to deform the fabric upon actuation.

In comparison with elastomeric actuators, the actuators described herein can offer very little to no resistance when deflated, as they are fabric-based. In contrast, an elastomer actuator, when depressurized, can still be difficult to bend due to a need to strain the elastomer. Consequently, the actuators described herein can be very nonrestrictive when worn but can also provide force or stiffen considerably when pressurized. Additionally, when pressurized, the actuators described herein can stiffen and take a preformed shape, which can advantageous for some bracing applications.

### BRIEF DESCRIPTION OF THE DRAWINGS

The invention is described with reference to the following figures, which are presented for the purpose of illustration only and are not intended to be limiting. In the Drawings:

FIG. 1 presents exploded and assembled views of a fiber-reinforced soft actuator 10 and the components that compose (A) bending, (B) bend-twist, (C) extend, (D) and extend-twist actuators, with illustrations of inactive (non-pressurized, at left or top) and active (pressurized, at right or bottom) states.

FIG. 2 presents an illustration of a plain weave structure 18 with the warp extending vertically and weft extending horizontally in the orientation shown.

FIG. 3 presents an illustration of a weft knit structure 24′ with the wales extending vertically and the weft and course extending horizontally in the orientation shown.

FIG. 4 presents an illustration of a warp knit structure 24″ with the wales and warp extending vertically and courses extending horizontally in orientation shown. FIG. 5 compares the load-extension behaviors along the X and Y axis of an isotropic woven fabric material 18 and an anisotropic knit fabric material 24.

FIG. 6 presents a sample one-way stretch knit material 24 anchored along one edge.

FIG. 7 presents the sample knit material 24 from FIG. 6 stretched with a force, F, along the X-direction.

FIG. 8 presents the sample knit material 24 from FIG. 6 stretched with a force, F, along the Y-direction.

FIG. 9 presents a sample knit material 24 with inextensible thread 30 straight stitched into the sample 24 and a force, F, applied in-line with the inextensible thread 30.

FIG. 10 presents a sample knit material 24 with inextensible thread 30 zig-zag stitched into the sample 24.

FIG. 11 depicts the sample 24 in FIG. 10 being stretched with a force, F, until the zig-zag stitches 30 are straight and limit further extension.

FIG. 12 depicts a knit material 24 with an inextensible thread 30 straight stitched at an angle.

FIG. 13 presents a knit material 24 with a combination of straight and zig-zag stitches 30′ and 30″.

FIG. 14 depicts a force, F, evenly applied to the right edge of the fabric 24 in FIG. 13, where the stitches 30 limit the stretch response of the material 24 by different amounts.

FIG. 15 is a perspective view of a material layer 32 folded to form sequential knife pleats 34.

FIG. 16 is a side view of the pleated material layer 32 of FIG. 15, depicting pleat depth, L₁, and pleat spacing, L₂.

FIG. 17 is a top view of the pleated material layer 32 of FIG. 16.

FIG. 18 depicts a side view of the length change of the pleated material 32 in FIG. 16 as the material is unfolded.

FIG. 19 depicts a top view of the length change of the pleated material 32 in FIG. 16 as the material is unfolded.

FIG. 20 presents a top view of a pleated material 32, where the pleats 34 are oriented at an angle.

FIG. 21 presents an exploded view of a fabric-based fluidic actuator 10, including a first fabric layer 42, a bladder 36 coupled with and in fluid communication with a pressurized fluid line 38, and a second material layer 44.

FIG. 22 presents an exploded side view of the components for a fabric-based fluidic actuator 10.

FIG. 23 presents a depiction of a bending fabric-based fluidic actuator 10 assembled with a straight stitch 30 along its perimeter.

FIG. 24 presents a fluid-pressurized, bending, fabric-based actuator 10.

FIG. 25 presents a prototyped, fluid-pressurized, bending, fabric-based actuator 10, where the first fabric layer 42 is a pleated one-way stretch knit 24, and where the second material layer 44 is an inextensible woven layer 18.

FIG. 26 presents a side view of the components of a bending actuator 10 with a first fabric layer 42 (here, a pleated layer 32) coated with a stretchable low-gas-permeable plastic 40 and a second fabric layer 44 coated with stretchable low-gas-permeable plastic 40, wherein the low-gas-permeable plastic functions as the bladder.

FIG. 27 presents a prototyped, fluid-pressurized, bending, fabric-based actuator 10, where the first fabric layer 42 is a one-way stretch knit 24, and where the second fabric layer 44 is an inextensible woven layer 18.

FIG. 28 presents a prototyped, fluid-pressurized, bending, fabric-based actuator 10, where the first fabric layer 42 is a one-way stretch knit 24 reinforced with straight stitches 30, and where the second material layer 44 is an inextensible woven layer 18.

FIG. 29 depicts a fluid-pressurized, bend-twist, fabric-based actuator 10, where the first fabric layer 42 has pleats 34 or reinforcements (e.g., stitches 30 of an inextensible thread) that are oriented at a non-zero angle to the length and width of the fabric.

FIG. 30 presents a depiction of a bend-extend, fabric-based, fluidic actuator 10 assembled with a zig-zag stitch 30 along its perimeter, where the first fabric layer 42 is pleated and is designed to stretch more than the second material layer 44, which is composed of a one-way stretch knit fabric 24.

FIG. 31 depicts an extension in length of a bend-extend actuator 10 over the bending actuator of FIG. 24.

FIG. 32 presents an assembled side view of an unpressurized fabric-based actuator 10, where the material layers 42 and 44 promote nearly the same length extension (ΔL) upon pressurization.

FIG. 33 depicts extension of the fabric-based actuator 10 of FIG. 32 after pressurization.

FIG. 34 demonstrates the twist-extend response of the fabric-based actuator 10 when the material layers 42 and 44 are joined with a zig-zag seam 45 and angled pleats 34 and appropriately oriented at a non-zero angle to the length and width of the fabric.

FIG. 35 presents stitch reinforcements 30 on the first material layer 42 that permit different amounts of stretch in the Y-direction.

FIG. 36 depicts the first material layer 42 from FIG. 35 applied to a fluid-pressurized bending actuator 10, where the zig-zag stitch reinforcements 30″ (shown here stretched out) influence the actuator's cross-sectional area.

FIG. 37 presents straight stitch reinforcements 30 on the first material layer 42 that permit different amounts of stretch in the X- and Y-directions.

FIG. 38 depicts the first material layer 42 from FIG. 37 connected to an inextensible second fabric layer 44, where the stitch reinforcements 30 on the first material layer 42 restrict bending at the actuator ends and permit bending at a section 47 in the middle of the actuator 10.

FIG. 38 presents a first material layer 42 composed of woven materials 18 connected to a section of pleated knit material 32.

FIG. 40 presents a side view of a pressurized actuator 10 with the first fabric layer 42 in FIG. 39 connected to an inextensible second fabric layer 44.

FIG. 41 presents an exploded side view of the components for a fabric-based fluidic actuator 10 with segments of stiff inclusions 46.

FIG. 42 presents an assembled isometric view of the actuator 10 of FIG. 41, where the textile actuator 10 includes pockets 48 into which stiff inclusions 46 can be inserted or removed.

FIG. 43 presents a side view of the actuator 10 of FIG. 42 pressurized.

FIG. 44 presents a perspective of an actuator 10 with a stiff inclusion 46 attached to the outside of the actuator body.

FIG. 45 presents a side view of the actuator 10 of FIG. 44 in a pressurized state.

FIG. 46 presents an isometric view of an actuator body with a tapered profile.

FIG. 47 presents a side view of the actuator 10 in FIG. 46 pressurized.

FIG. 48 presents an exploded view of the components of a bi-morph bending actuator 10.

FIG. 49 depicts an assembled side view of the bi-morph bending actuator 10 of FIG. 48 and the range of motion of a bi-morph bending actuator 10 when the two bladders 36 are inflated separately and together.

FIG. 50 presents a side view of a fabric-based fluidic actuator 10 where the second bladder 36″ inflates to a rigid beam when pressurized while the first bladder 36′ can create bending.

FIG. 51 presents an alternative reinforcement method where a reinforcing (strain-limiting) material 50 is printed on and adheres to the textile material 42/44.

FIG. 52 presents a cross-section of the reinforcing material 50, where the material core 52 can be a strain-limiting material or a strain-sensing material.

FIG. 53 is an illustration of an actuatable shoulder and torso harness (vest) 54 incorporating bladders 36 between fabric layers 42 and 44 for actuation.

FIG. 54 depicts the harness 54 of FIG. 53 upon actuation (with pressurized bladders 36).

FIG. 55 illustrates a leg brace 56 including a plurality of bending actuators 10 embedded in fabric layers 58 and a rigidizing beam 60.

FIG. 56 presents an application of fabric-based fluidic actuators 10 adapted to a glove 62 to support hand opening and closing.

FIG. 57 presents an illustration of hand closure around an object 64 via actuation of the support glove 62 of FIG. 56.

FIGS. 58-60 show a Merrow seam 45 joining a first fabric layer 42 and a second material layer 44 with an in-the-round fabric with the flat seam 45 shown in FIG. 60.

FIG. 61 shows a pleated actuator 10, wherein the fabric wales extend along axis x, while the fabric courses and alignment of the seam 45 extend along axis y.

FIG. 62 shows the layered structure of thus actuator 10 of FIG. 61, illustrating the Merrow stitch that forms the seam 45, a high-stretch pleated knit first layer 24a/32/42 a thermoplastic elastomer (TPE) balloon that forms the bladder 36, and a low-stretch knit layer 24b/44.

FIG. 63 is a photographic image of an embodiment of the actuator 10 of FIG. 61 while actuated via pressurization.

FIG. 64 is a top view of a segmented pleated actuator 10 with a Merrow seam and with pleated sections of a high-stretch knit fabric 24a/32 interspersed low-stretch knit fabric sections 24b, which together form the first fabric layer 42.

FIG. 65 is a bottom view of the actuator 10 of FIG. 64 showing the relatively inextensible second material layer 44.

FIGS. 66 and 67 are photographic images of an embodiment of the actuator 10 of FIGS. 64 and 65 when actuated via pressurization.

FIGS. 68 and 69 show embodiments of the actuator 10, wherein the segmentation of the first fabric layer 42 into high-stretch knit fabric sections 24a with adjacent low-stretch knit fabric sections 24b can be along a longitudinal axis (in FIG. 68) and along an axis orthogonal to the longitudinal axis (in FIG. 69).

FIGS. 70 and 71 show an embodiment of a segmented textile actuator with a Merrow seam, wherein both the first fabric layer 42 (shown in FIG. 70) and the second material layer 44 (shown in FIG. 71) are segmented knit fabrics, wherein the first fabric layer 42 includes sequential segments of a no-stretch woven structure 25 and a high-stretch knit structure 24a, while the second material layer 44 includes sequential segments of a no-stretch woven structure 25 and a low-stretch knit structure 24b.

FIGS. 72 and 73 are photographic images of an embodiment of the actuator 10 of FIGS. 70 and 71, when actuated via pressurization.

FIGS. 74-76 show a fully pleated actuator 10 with a Merrow seam, wherein the first fabric layer 42 is a pleated high-stretch knit fabric 24a/32, while the second material layer 44 is a low-stretch knit fabric 24b. When actuated via pressurization, the actuator 10 curls into a coiled structure, as shown in FIG. 76.

FIGS. 77-79 show a full-gathered actuator 10 with a Merrow seam, wherein the first fabric layer 42 is a high-stretch gathered knit (as seen in FIG. 77), while the second material layer 44 is a low-stretch knit (as seen in FIG. 78). As seen in FIG. 79, the actuator 10 wraps into a coil when actuated via pressurization.

FIGS. 80-82 show an articulated actuator glove 70 including actuators 10 on a human hand 72.

FIGS. 83-86 show a gathered actuator 10 with a Merrow seam and segments of gather separated by low-stretch fabric sections 24b; FIG. 84 is a photographic image of this actuator 10 actuated via pressurization. Photographic images of a first fabric layer 42 with gathered segments 74 are also provided in FIGS. 85 and 86.

FIGS. 87-89 show a segmented gathered actuator 10 with a Merrow seam, wherein the first fabric layer 42, here with sequential segments of a one-way stretch knit 24′ and a gathered high-stretch knit 24a/32, is shown in FIG. 87. The second material layer 44, which is formed of the one-way stretch knit 24′ is shown in FIG. 88. A photographic image of this actuator 10 under pressure actuation is shown in FIG. 89.

FIG. 90 shows a front view of a human wearing a deflated shoulder support 76 and a deflated elbow support 78, each of which includes a plurality of the fabric actuators 10.

FIG. 91 shows a back view of the human wearing the deflated shoulder support 76, as seen in FIG. 90.

FIG. 92 shows a front view of the human wearing the shoulder support 76, as seen in FIG. 90, in an inflated state.

FIG. 93 shows a front view of the human wearing the elbow support 78, as seen in FIG. 90, in an inflated state.

FIGS. 94 and 95 show a human wearing a hip support 80′ (deflated) and 80″ (inflated), a knee support 82′ (deflated) and 82″ (inflated), and an ankle support 84′ (deflated) and 84″ (inflated).

FIGS. 96 and 97 respectively show a front and side view of a soft inflatable lung-diaphragm assistance vest 86 worn on a human torso, providing abdomen and chest support to facilitate breathing.

FIGS. 98 and 99 respectively provide a front and side view of stiffening full-leg-support pants 88 worn by a human in a deflated state (FIG. 98) and in an inflated state (FIG. 99).

FIGS. 100-102 show a human wearing a soft inflatable vest 86 on the torso for upper body (back and chest) support on impact, with FIG. 100 showing a front view, FIG. 101 showing a back view, and FIG. 102 showing a side view with the actuators 10 inflated.

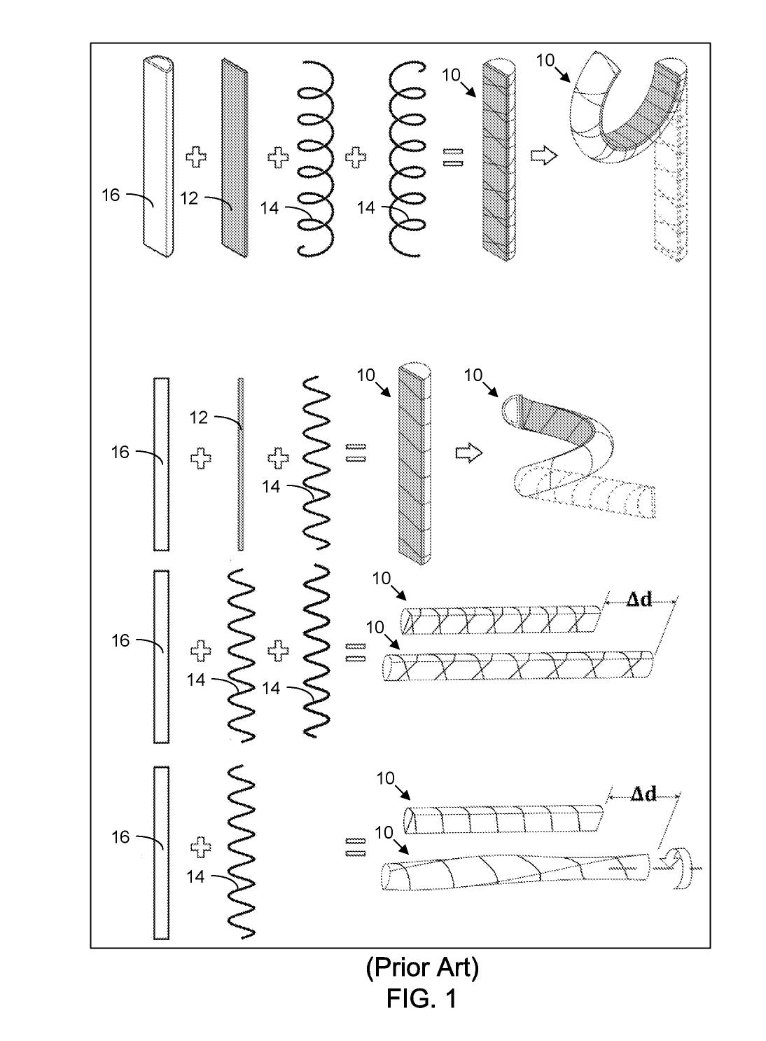

In the accompanying drawings, like reference characters refer to the same or similar parts throughout the different views; and apostrophes and letters are used to differentiate multiple instances of the same or variations of items sharing the same reference numeral. The drawings are not necessarily to scale or shape; instead, an emphasis is placed upon illustrating particular principles in the exemplifications discussed below.

### DETAILED DESCRIPTION

The foregoing and other features and advantages of various aspects of the invention(s) will be apparent from the following, more-particular description of various concepts and specific embodiments within the broader bounds of the invention(s). Various aspects of the subject matter introduced above and discussed in greater detail below may be implemented in any of numerous ways, as the subject matter is not limited to any particular manner of implementation. Examples of specific implementations and applications are provided primarily for illustrative purposes.

Unless otherwise herein defined, used or characterized, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially (though not perfectly) pure, as practical and imperfect realities may apply; e.g., the potential presence of at least trace impurities (e.g., at less than 1 or 2%) can be understood as being within the scope of the description. Likewise, if a particular shape is referenced, the shape is intended to include imperfect variations from ideal shapes, e.g., due to manufacturing tolerances. Percentages or concentrations expressed herein can be in terms of weight or volume. Processes, procedures and phenomena described below can occur at ambient pressure (e.g., about 50-120 kPa—for example, about 90-110 kPa) and temperature (e.g., −20 to 50° C.—for example, about 10-35° C.) unless otherwise specified.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments.

Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly.

Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms, “includes,” “including,” “comprises” and “comprising,” specify the presence of the stated elements or steps but do not preclude the presence or addition of one or more other elements or steps.

Additionally, the various components identified herein can be provided in an assembled and finished form; or some or all of the components can be packaged together and marketed as a kit with instructions (e.g., in written, video or audio form) for assembly and/or modification by a customer to produce a finished product.

Described herein are fabric-based fluidic actuators made by bonding two or more material layers to form a pocket and positioning or integrating a bladder configured to hold pressurized fluid inside the pocket. A stretchable fabric layer, as discussed herein, may refer to knit fabrics, such as one-way or two-way stretch; warp knit and weft knit fabrics; knit woven, and nonwoven fabrics modified with stitch reinforcements; knit, woven, and nonwoven fabrics modified with segments of bonded materials; knit, woven, and nonwoven fabrics manufactured with anisotropic properties; pleated knits, gathered knits, and pleated and gathered woven and nonwoven fabrics; or stretch woven fabrics in which a yarn made from an elastic fiber is used in at least one orientation. A bladder, as used herein, includes a pouch constructed from, e.g., plastic film and connected to a pressurized fluid source. The bladder can be thinner [e.g., less than 0.2-mm thick—for example 1.5 mils (˜0.04 mm) thick] and lighter weight (even with the added weight of the fabric) than previous elastomeric soft actuator bodies, which typically were much thicker and heavier. Non-limiting examples of the composition of the plastic film include elastic polymer (e.g., urethanes and silicones), thermoplastic elastomers (TPEs), thermoplastic urethanes (TPUs), heat-sealable rip-stop nylon, polytetrafluoroethylene (PTFE), etc. The bladder can be a discrete structure separate from the fabric/material layer, or the bladder can be integrated with (e.g., coated on, impregnated into or laminated or heat-bonded onto) the fabric/material layers.

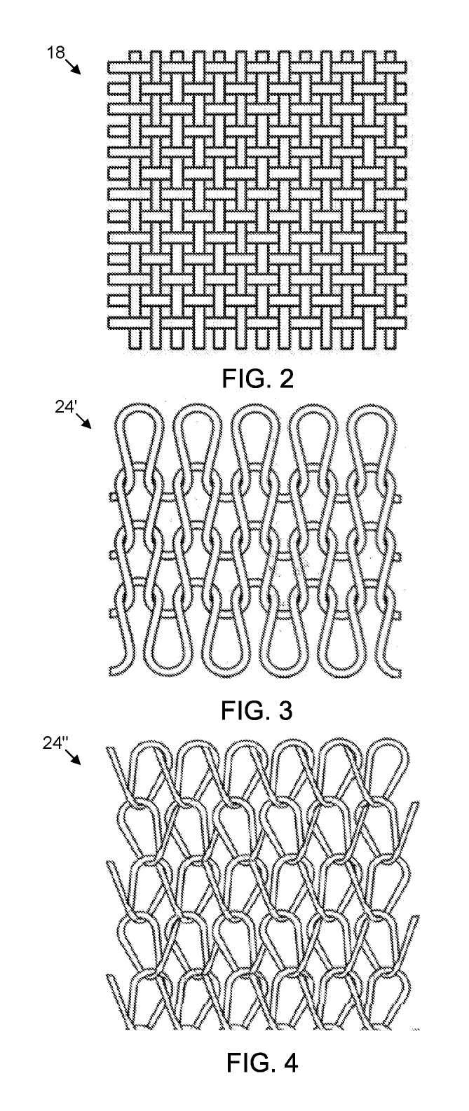

The shape and range of motion of a fabric-based fluidic actuator depends in large part on the anisotropic properties of the material layers under tension. Unlike other materials traditionally used for engineering applications, such as metal and rigid plastics, fabrics differ considerably because they are not continuous and instead are formed of a network of fibers or yarn extending along different directions. The mechanical properties of the fibers and the method used to construct the network of fibers (such as knitting or weaving) can change the global properties of the fabric significantly. Two network construction techniques that can lead to significantly different behavior of the fabric are (a) weaving and (b) knitting. A plain weave construction 18 (as shown in FIG. 2) with inextensible fibers leads to inextensible behavior in the warp (vertical) and weft (horizontal) direction of the fabric 18. Some motion is possible along the bias (a direction that is not aligned with the warp and weft direction); however, if loads are distributed along the warp and weft directions, the fabric 18 will be relatively inextensible. Examples of commercially available woven inextensible fabric that may be used include nylon rip-stop fabric, vinyl-coated woven polyester fabric, and woven cotton/polyester fiber blends.

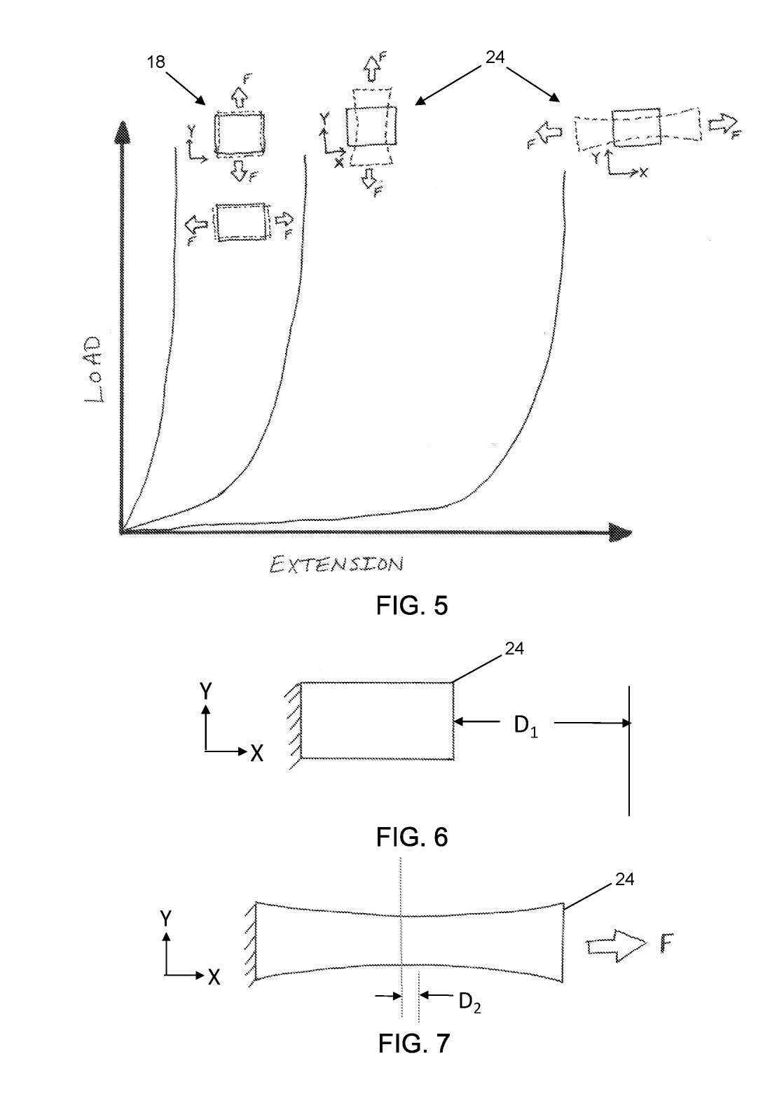

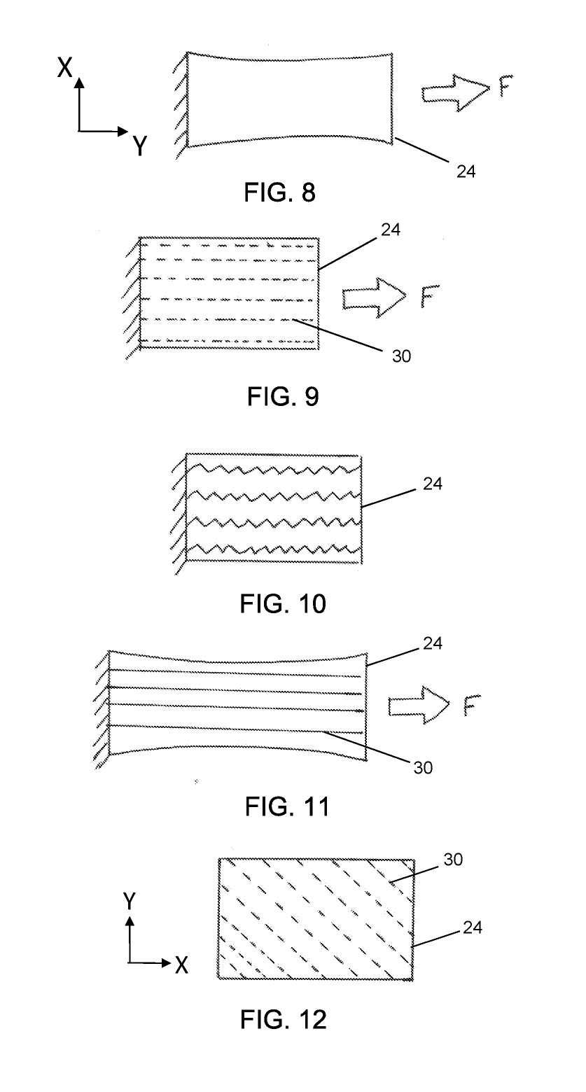

Alternately, a knit construction 24, which can have a weft knit structure (as shown in FIG. 3) or a warp knit structure (as shown in FIG. 4), may allow the fabric to stretch even when the fibers are inextensible. Commercially available fabrics utilize a combination of material and construction variations to develop fabrics with different behaviors. Thus, woven and knit fabric layers 18 and 24 can have different load-extension properties depending on the direction of the load in relation to the threads or fibers that compose the fabric layer. The load-versus-extension plot of FIG. 5 illustrates this principle, where an isotropic material, such as a woven fabric 18 may have roughly the same load-extension response whether the fabric is loaded along the X-axis or Y-axis. Anisotropic fabrics, such as one-way stretch knit fabrics 24, may have properties built into the fabric during the manufacturing process, where the load-extension response along the Y-axis may be very different from the load-extension response along the X-axis, as shown in the two right-most plots of FIG. 5. FIGS. 6-8 illustrate this property more clearly, where FIG. 6 presents an unloaded knit fabric layer 24 anchored on one side. FIG. 7 presents one configuration where the knit fabric layer 24 stretches by an amount, D₁, when a force is applied to the opposing side. FIG. 8 presents the same knit fabric layer 24 when its orientation is changed, where its extension with the same force is smaller (D₂<D₁).

In some embodiments, the load-extension response of a knit fabric layer 24 can be modified by adding a strain-limiting material. In one specific embodiment, several straight locking stitches 30 composed of threads or extensible fibers can be added, as shown in FIG. 9, to the knit fabric layer 24. The threads in the stitch 30 support the load and prevent the knit fabric layer 24 from stretching. Stitched fibers 30 allow local control over the direction of reinforcements on the fabric 24, and this technique can be useful for developing a wide range of anisotropic properties (and resulting actuator motions) by modifying the layout of the stitched fibers 30 on the base fabric 24. In FIG. 9, the stitch reinforcements 30 can be designed to have minimal to no influence on the stretch properties of the knit fabric layer 24 in the Y-direction. In another embodiment, reinforcements that allow stretch, such as zig-zag, flatlock, interlock, overlock stitches 30 or a sewn segment of applied or bonded material, can be added to the knit fabric layer 24, as shown in FIG. 10. Stretchable stitches 30, such as a zig-zag stitch, offer the advantage that they permit the knit fabric layer 24 to stretch until the threads in the stitch 30 become taut (as shown in FIG. 11), until the fabric 24 stretches to its limit, or until the force from the pressurized bladder 36 balances with the strain-limiting and stretch properties of the knit fabric layers 24.

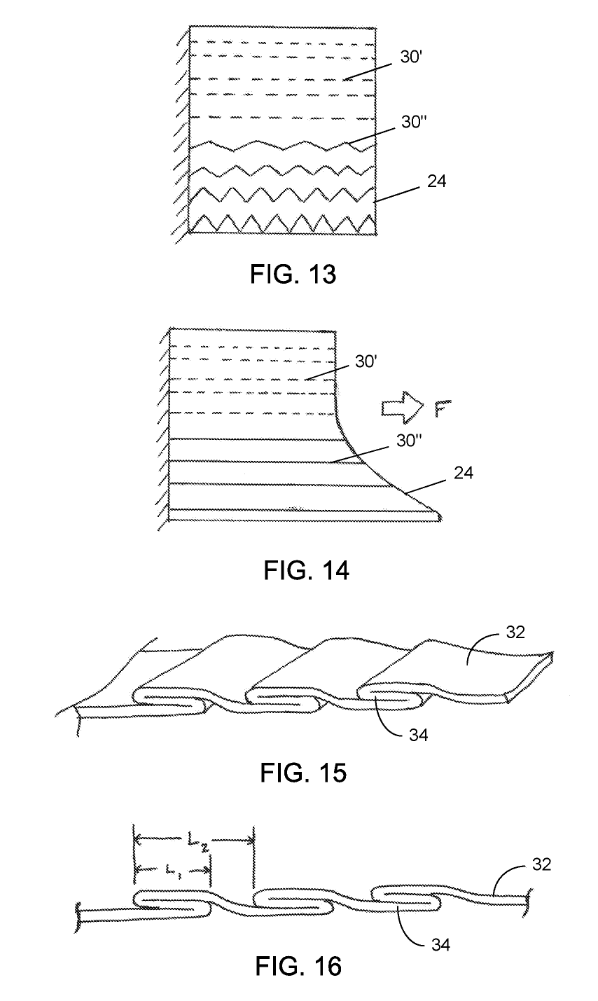

In some embodiments, these orientation, spacing, and strain-limiting strategies can be combined in various ways to generate specific anisotropic properties in a knit fabric layer 24. In a specific embodiment, the stitch reinforcements 30 can be added at different angles relative to the loading direction, as shown in FIG. 12. In another specific embodiment, straight and zig-zag stitches 30′ and 30″ can be combined in parallel, as shown in FIG. 13. In this example, the amplitude of the zig-zag stitches 30″ vary as a function of length down the sample. A load evenly applied along the bottom edge of this knit material layer 24 results in sections that do not stretch and a section that can exhibit a gradient stretch response, as shown in FIG. 14. Furthermore, stitch reinforcements 30 can be combined in a multitude of other configurations. For example, straight stitches 30′ and zig-zag stitches 30″ can be combined in series. In another example, stitch reinforcements 30 can be combined at different angles and intersect with each other to alter the knit material layer's load-extension.

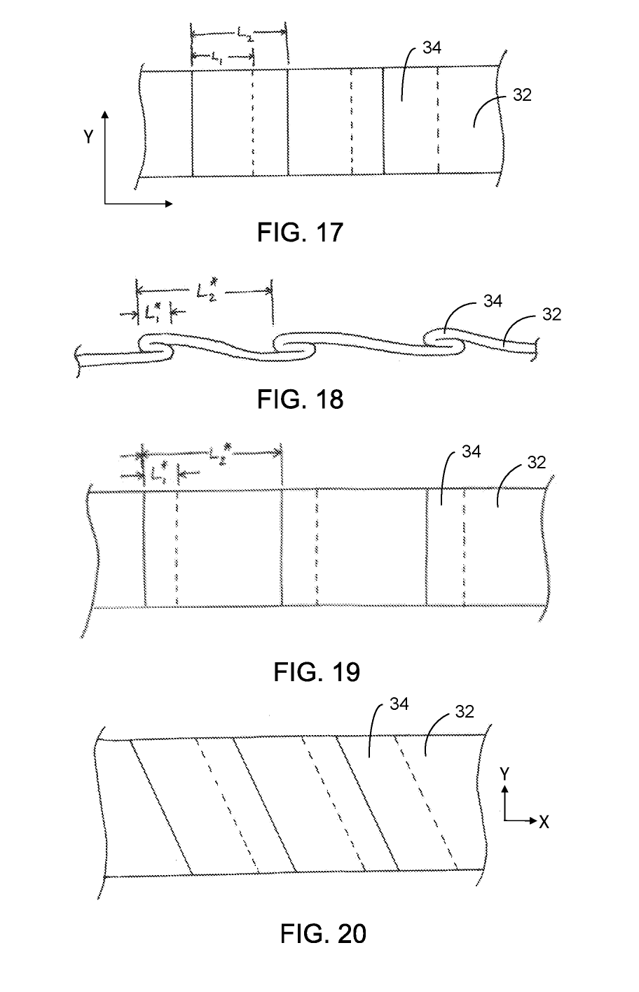

In another embodiment, the anisotropic properties of a material layer can be modified by pleating the material. FIG. 15 presents an isometric view of a material (e.g., fabric) layer 32 that has successive folds or knife pleats 34. FIGS. 16 and 17 present side and top views, respectively that illustrate some of the pleating variables including the pleat depth, L₁, and the pleat spacing, L₂. A notable feature of a material layer 32 composed of pleats 34 is that length extension occurs through unfolding, and unfolding can require very low forces. In other words, pleated material layers 32 can be designed to produce large changes in length with minimal force. FIGS. 18 and 19 illustrate the length extension of a pleated material layer 32 through unfolding where the pleat depth, L₁*, and the pleat spacing, L₂*, increases. It should be noted that while pleated material layers 32 enable length extension with minimal force in one direction (i.e., the X-direction, as depicted in the FIGS. 15-19), the pleats 34 can increase resistance to extension in another direction (i.e., the Y-direction as depicted in FIGS. 15-19). This result is because the pleats 34 enable more material to be added per unit length, which can increase a material layer's resistance to extension compared to a non-pleated material layer. Furthermore, as described above, with respect to the stitch-reinforced material, a variety of parameters, such as pleating orientation (e.g., as shown in FIG. 20), spacing, and pleat method (e.g., box pleat, double box pleat, rolled pleat, sunary pleats and dart pleats) or scrunching or gathering can be combined in various ways to generate a range of anisotropic properties in a pleated material layer 32.

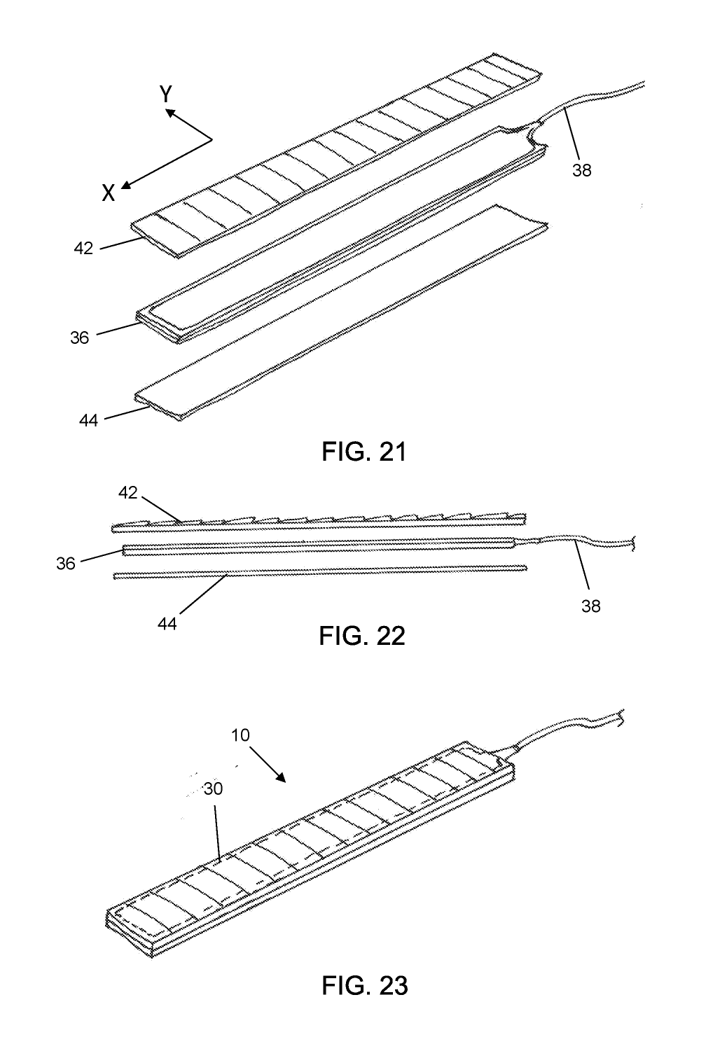

In many methods and combinations, material layers can be assembled to create fabric-based fluidic actuators. The embodiment of FIGS. 21 and 22 present an exploded isometric and side view, respectively, of the components for building a fabric-based actuator 10 that bends upon fluid pressurization. The first fabric layer 42, which is a pleated layer 32 here, is an anisotropic layer oriented with minimal resistance to stretch along the longitudinal direction (X-direction) and greater resistance to stretch along the radial direction (Y-direction). The second material layer 44 preferably has high resistance to stretch (similar to the isotropic material in FIG. 5)—i.e., is strain-limiting—but is still flexible. Example materials for the second material layer 44 include woven and non-woven fabrics. The first fabric layer 42 and second material layer 44 are joined together either by sewing or other bonding methods.

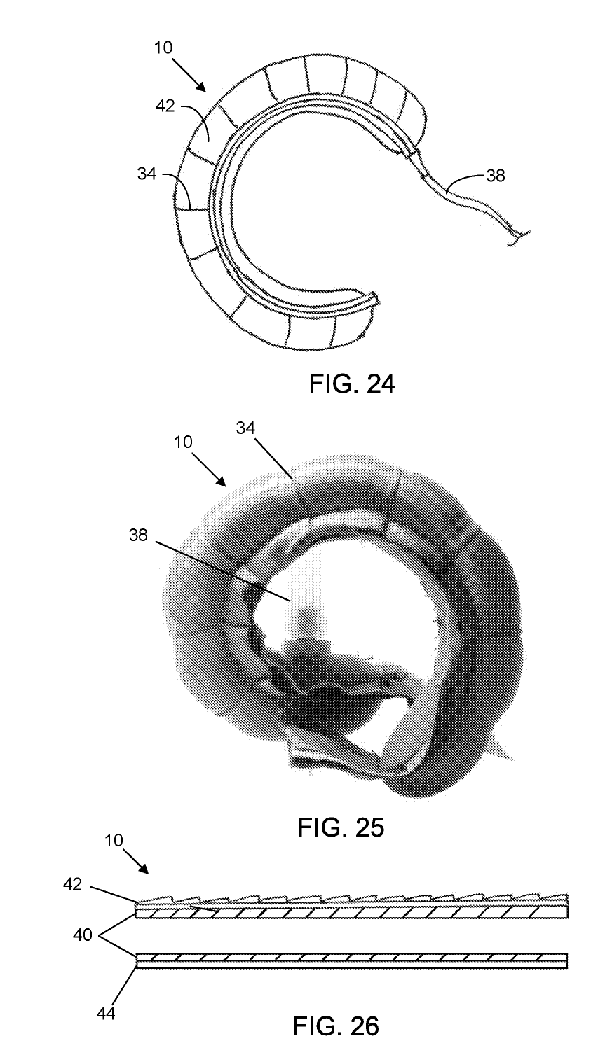

In FIG. 23, a straight stitch 30 along the perimeter joins the layers 42 and 44 together and creates a cavity for a bladder 36 (shown, e.g., in FIG. 2′). The bladder 36 can be placed between the two layers 42 and 44 and sewn together with them or inserted in the cavity after the material layers 42 and 44 are sewn together. The primary function of the bladder 36 in this and other embodiments is to hold pressurized fluid (e.g., air or another gas or liquid) and, advantageously, can be larger than the cavity defined by the two material layers 42 and 44, such that, in all configurations of the fabric-based actuator 10, the material layers 42 and 44 are stressed more than the bladder 36. When the bladder 36 is pressurized, the material layers 42 and 44 experience tension both longitudinally and circumferentially. Bending motion corresponds to a longitudinal stretching of the first fabric layer 42 while the second material layer 44 does not undergo any transformation. Therefore, an actuator 10 that includes a first fabric layer 42 that preferentially stretches in the longitudinal direction and a second material layer 44 that is inextensible will be kinematically consistent with the motion of a bending actuator 10. It should be noted that material layers with greater resistance to stretching in the Y-direction or circumferentially will expand less circumferentially at higher operating pressures. FIG. 24 presents a line drawing of a pressurized bending actuator 10, while a photograph of a prototyped bending actuator 10 with a pleated first fabric layer 32/42 and a woven inextensible second material layer 18/44 is provided in FIG. 25.

While FIG. 22 presents a construction method wherein a bladder 36 is disposed between two fabric layers 42 and 44 to create an actuator 10, an alternative method is presented in FIG. 26, where a low-gas-permeable (or gas-impermeable) plastic layer 66 coats (or is laminated to) the inside surfaces of the fabric layers 42 and 44 to create an actuator 10. In this approach, the plastic layer 66, which can be a thermoplastic elastomer, thermoplastic urethane, silicone, or polyurethane, can stretch and/or unfold with the fabric 42/44 while still maintaining gas impermeability or low-gas permeability. When the first fabric layer 42 and second material layer 44 are joined together either by sewing and/or with other bonding methods, such as thermal or chemical bonds, to create an air/water-tight perimeter, the plastic coating 66 forms the bladder 36 of the actuator 10. Furthermore, the coating 66 can be designed to not influence or to only minimally influence the load-extension mechanics of the fabric layer 42/44; thereby providing little mechanical value other than to hold pressurized fluid. The embodiments presented throughout this disclosure detail construction methods that use a bladder 36 separate from the fabric/material layers 42/44; however, these methods and apparatus can substitute the plastic-coated fabrics (where the plastic coating forms the bladder) or other structures (e.g., plastic-impregnated fabrics) where the bladder is integrated with the fabric for the discrete fabrics and bladder to achieve a similar result.

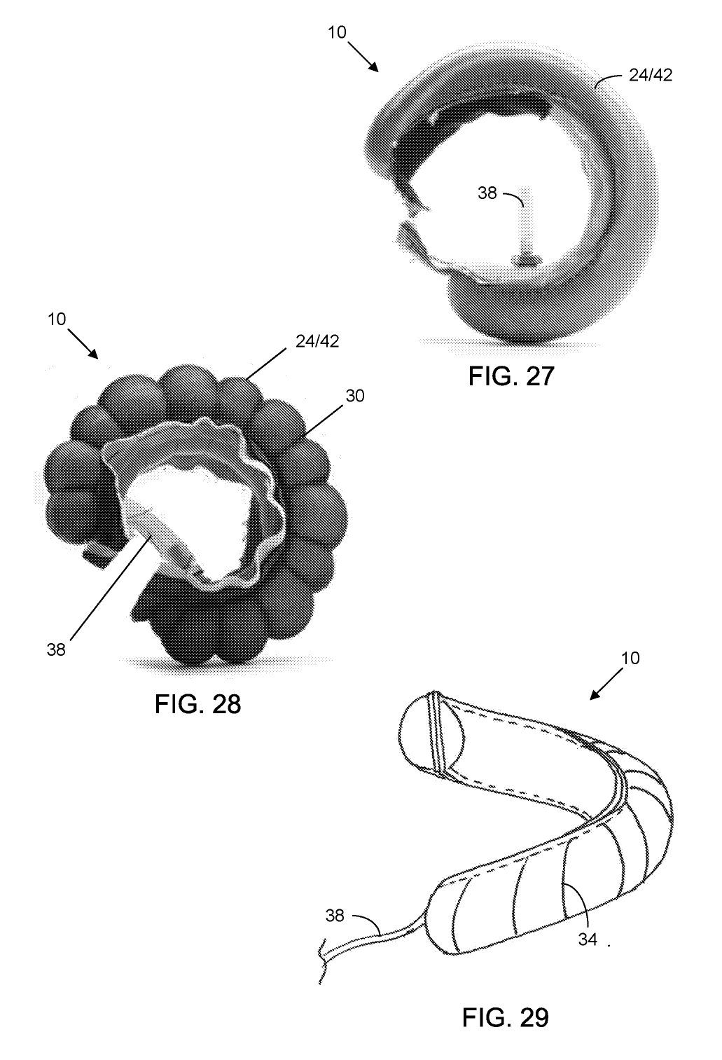

FIG. 27 presents a bending actuator 10, where a commercial off-the-shelf one-way stretch knit material 24′ was used as the first fabric layer 42. The one-way stretch knit 24′ has a low resistance to stretch along the longitudinal direction and more resistance to stretch circumferentially. FIG. 28 presents an example where the first fabric layer 42 has anisotropic properties similar to those of the actuator 10 presented in FIG. 9. In this example, a two-way stretch knit material 24″ was modified with straight stitches 30′ to preferentially limit the stretch of the two-way stretch knit material 24″ circumferentially and to minimally impede longitudinal stretch. The locations of the reinforcing stitches 30 on the first fabric layer 42 are evident by the crests and valleys that mark the profile of the pressurized actuator 10 in FIG. 28.

In another embodiment, the orientation of the first fabric layer 42 can be adjusted to produce a fabric-based actuator 10 that bends and twists upon fluid pressurization. For example, if the first fabric layer 42 has stretch properties similar to the fabric layers presented in FIG. 12 or FIG. 20, where the direction of minimal stretch resistance is angled, the actuator 10 will bend and twist to form a helical shape, as shown in FIG. 29. The pitch and bending radius of the helical shape is dependent on the stretch properties of the first and second fabric/material layers 42 and 44 and on the angle of the anisotropic properties of the first fabric layer 42. A wide range of motions can be achieved by changing the angle of the reinforcements. The angle can be consistent across the entire fabric layer or can vary. In scenarios where the angle is varied, a fabric-based actuator can be designed to bend for a portion and then bend-twist. Furthermore, the angle can be gradually increased (or decreased) to create a gradient that influences the resulting pitch of the bend-twist actuator 10.

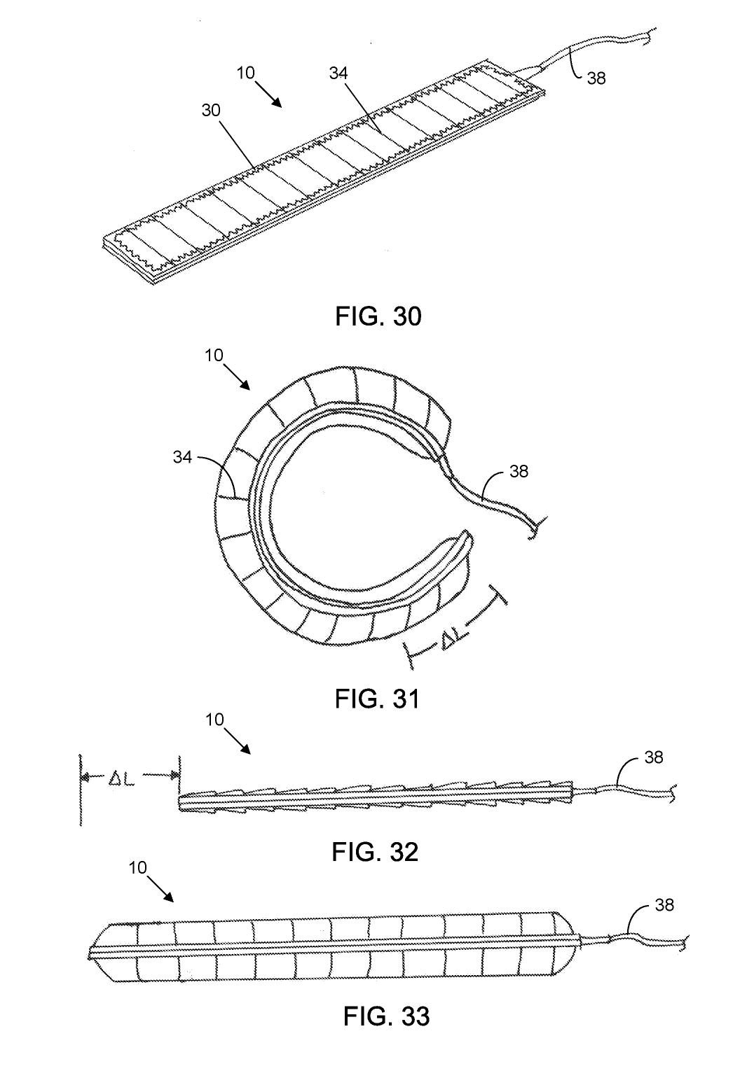

In another embodiment, fabric-based actuators 10 can be designed to bend and extend in length upon fluid pressurization. Following the construction method presented in FIGS. 21-23, the second material layer 44 can be replaced with an anisotropic material layer that has minimal resistance to stretch longitudinally and high resistance to stretch circumferentially (or in the Y-direction). The first fabric layer 42, however, can advantageously stretch more than the second material layer 44 to promote bending. Furthermore, the two layers 42 and 44 can be advantageously joined together with a bond or seam, such as a zig-zag stitch 30″, that permits stretch as shown in FIG. 30. Upon fluid pressurization of the bladder 36, the fabric-based actuator 10 will bend, and its length will increase by as much as is allowed by the seam. FIG. 31 depicts a bend-extend actuator 10 with ΔL marking the change in length over the bending depicted in FIG. 32.

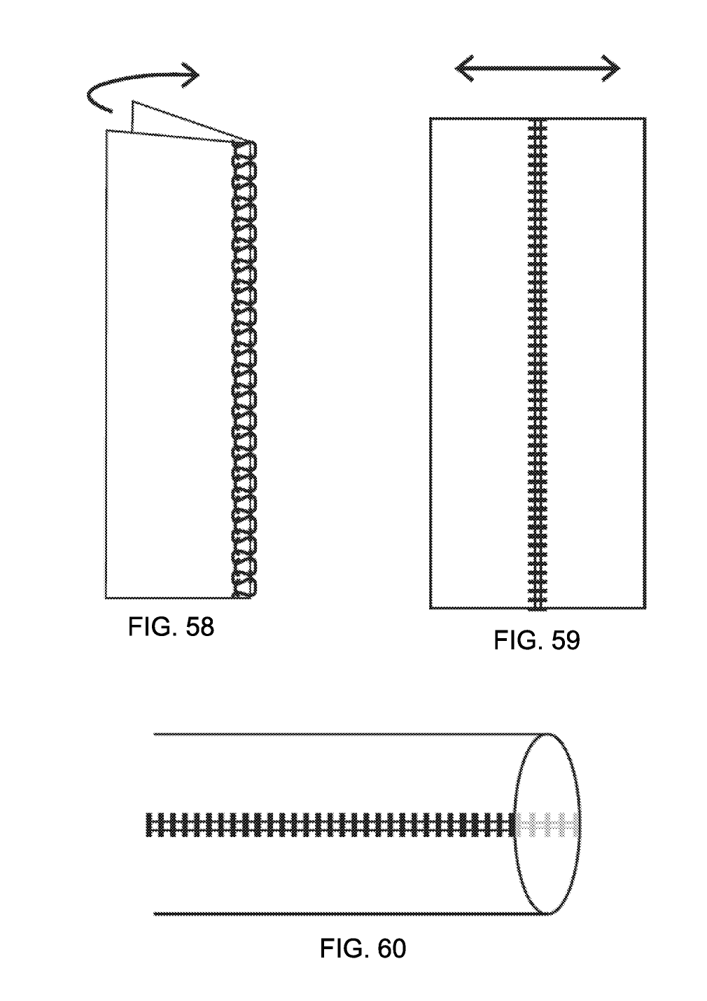

In additional embodiments, the seam can be a Merrow seam 45, as can be produced by a Merrow ACTIVESEAM MB-4DFO sewing machine from Merrow Sewing Machine Company (Fall River, Mass., US). A Merrow seam 45 joining a first fabric layer 42 and a second material layer 44 is shown in FIGS. 58-60. The Merrow seam 45 is seen joining the layers 42 and 44 along a salvage or cut edge in FIG. 58. The layers 42 and 44 are then opened apart as shown in FIG. 59 to produce a flat seam 45. An in-the-round fabric with the flat seam 45, which can be a single or double (or more) seam is shown in FIG. 60.

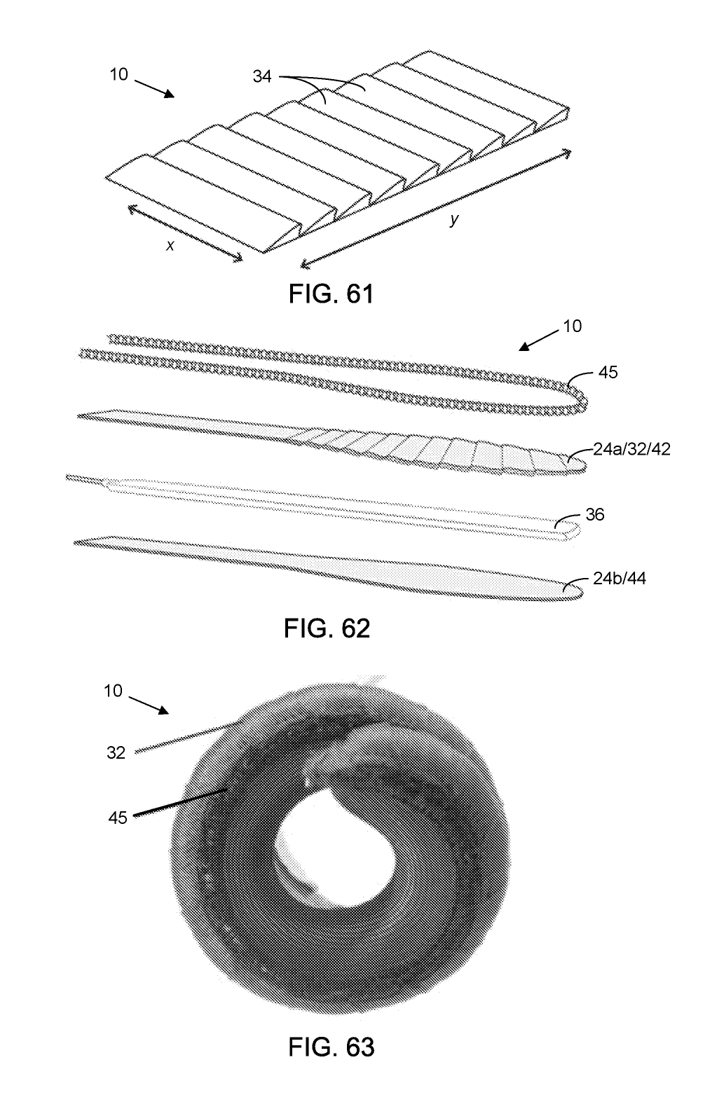

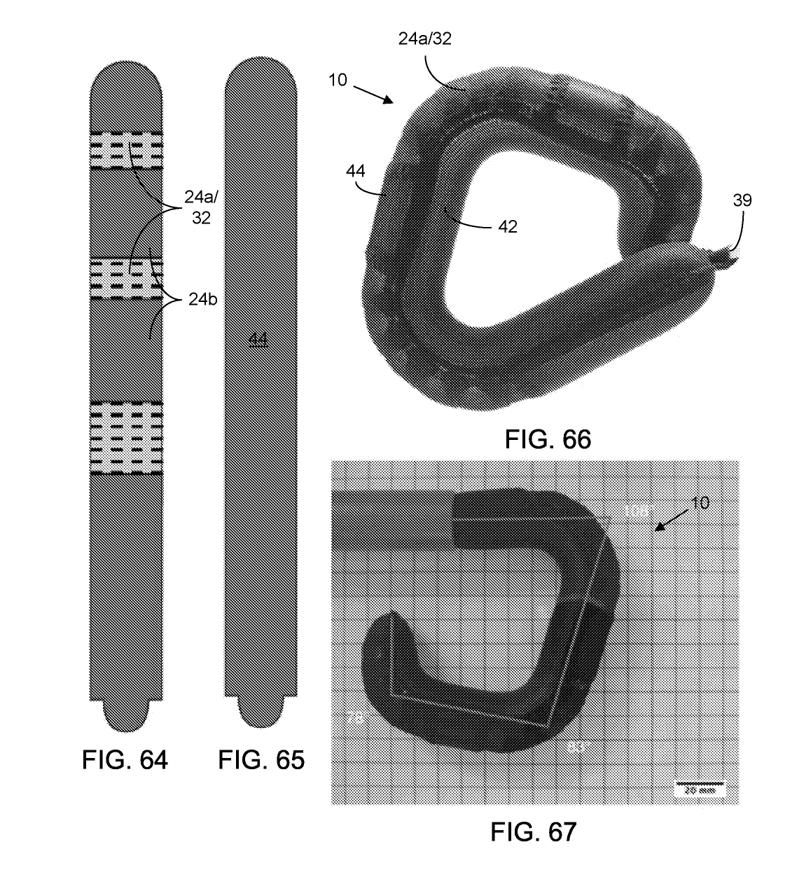

A pleated actuator 10 is shown in FIG. 61, wherein the fabric wales extend along axis x, while the fabric courses and alignment of the seam 45 extend along axis y. The layered structure of this actuator 10 is shown in the exploded image of FIG. 62, illustrating the Merrow ACTIVESEAM stitch that forms the seam 45, a high-stretch pleated knit first layer 24a/32/42 a thermoplastic elastomer (TPE) balloon that forms the bladder 36, and a low-stretch knit layer 24b/44. A photographic image of an embodiment of this actuator 10, wherein the actuator bends away from the pleated first layer 24a/32/42 upon pressurization as the actuator 10 is actuated. A top view of a segmented pleated actuator 10 with a Merrow seam and with pleated sections of a high-stretch knit fabric 24a/32 interspersed low-stretch knit fabric sections 24b, which together form the first fabric layer 42 is shown in FIG. 64. In alternative embodiments, a no-stretch woven fabric may be used in place of the low-stretch knit fabric 24b. The pleated high-stretch knit sections 24a/32 have greater elasticity than the low-stretch knit sections 24b, enabling the actuator 10 to bend (much like the joints of a finger) while the low-stretch knit sections 24b remain substantially rigid when the actuator is pressurized. A bottom view of the actuator 10 showing the relatively inextensible second material layer 44 is provided in FIG. 65. Photographic images of this actuator 10, when actuated via pressurization, are provided in FIGS. 66 and 67. As seen in FIG. 67, the “joints” of the actuator 10 (at the high-stretch sections) bend at 107°, 118°, and 127° in this example, though the specific angle in any particular embodiment will generally be determined by factors, such as the material properties, geometry and inflation pressure of the actuator 10.

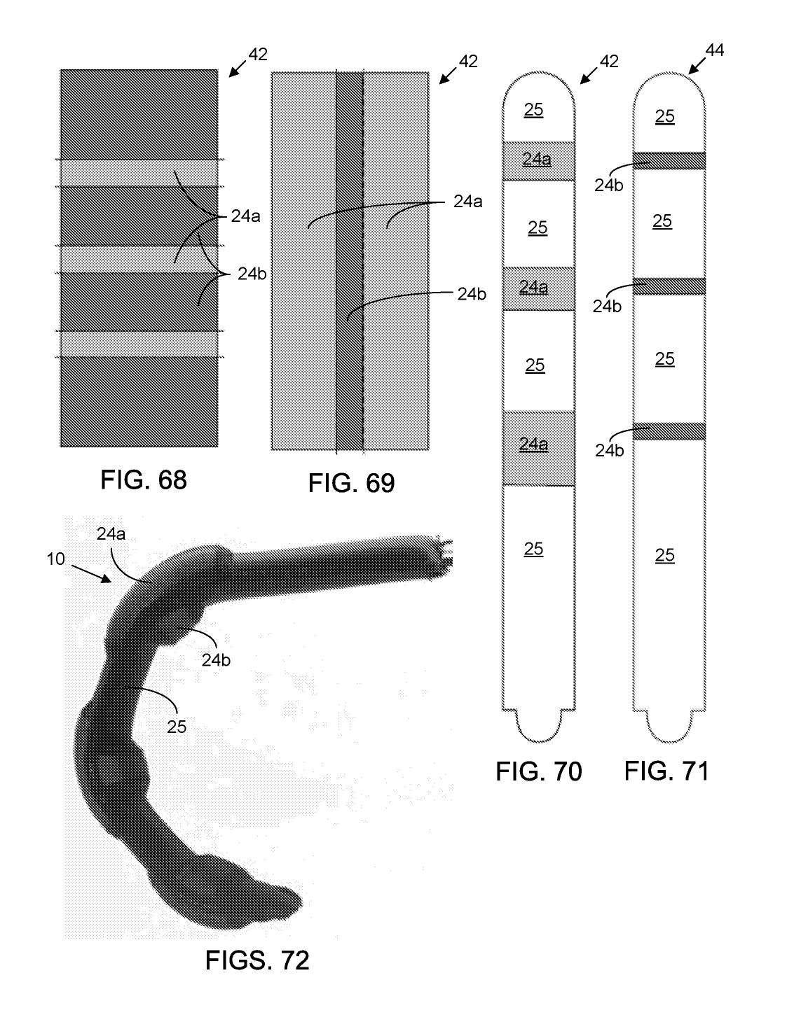

In various embodiments the segmentation of the first fabric layer 42 into high-stretch knit fabric sections 24a with adjacent low-stretch knit fabric sections 24b can be along a longitudinal axis (extending along the greatest dimension of the fabric), as shown in FIG. 68, or along an axis orthogonal to the longitudinal axis, as shown in FIG. 69.

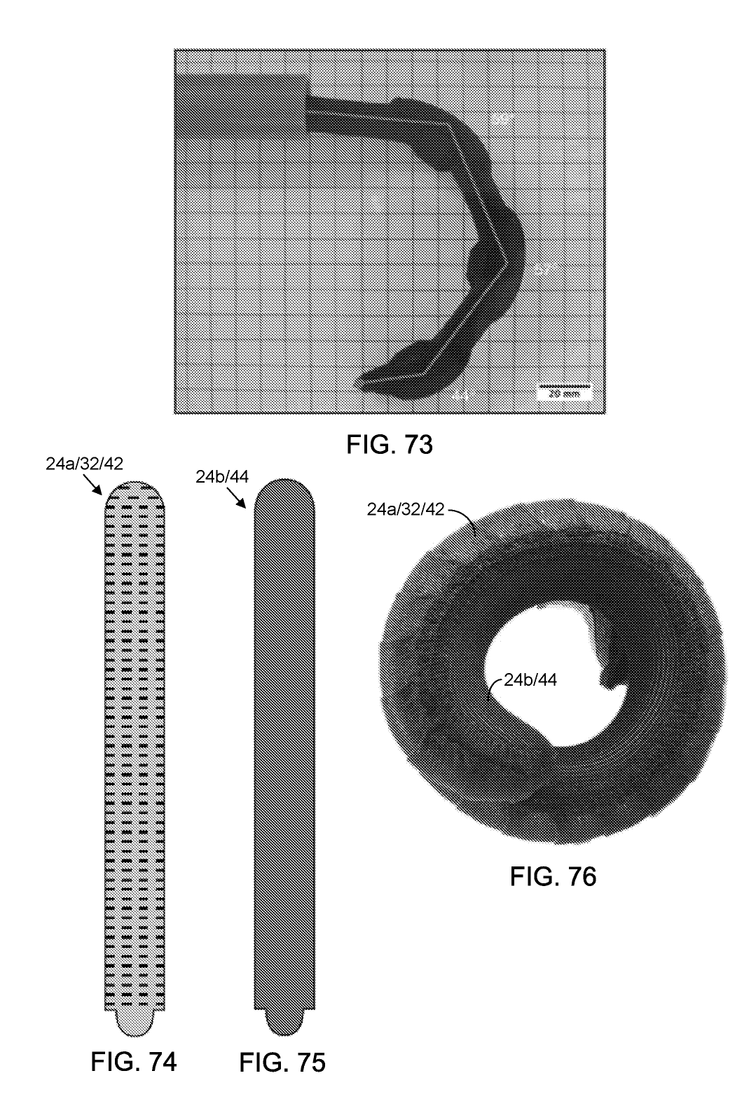

In yet another embodiment of a segmented textile actuator with a Merrow seam, both the first fabric layer 42, shown in FIG. 70, and the second material layer 44, shown in FIG. 71, are segmented knit fabrics. In this embodiment, the first fabric layer 42 includes sequential segments of a no-stretch woven structure 25 and a high-stretch knit structure 24a, while the second material layer 44 includes sequential segments of a no-stretch woven structure 25 and a low-stretch knit structure 24b, as shown in FIGS. 70 and 71, respectively. Photographic images of this actuator 10, when actuated via pressurization, are provided in FIGS. 72 and 73. As seen in FIG. 73, the “joints” of the actuator 10 (at the high-stretch/low-stretch sections) bend at 59°, 57°, and 44°. In an alternative embodiment, the low-stretch segments of the second material layer 44 can be omitted, such that the second material layer is formed entirely of a no-stretch woven structure 25; in this embodiment, the “joints” at the second material layer 44 may not be as clearly defined and the bending angles may be lower (e.g., 47°, 39°, and 22° at respectively corresponding joints in a similar actuator structure).

A fully pleated actuator 10 with a Merrow seam is shown in FIGS. 74-76. In this embodiment, the first fabric layer 42 is a pleated high-stretch knit fabric 24a/32, while the second material layer 44 is a low-stretch knit fabric 24b. When actuated via pressurization, the actuator 10 curls into a coiled structure, as shown in FIG. 76.

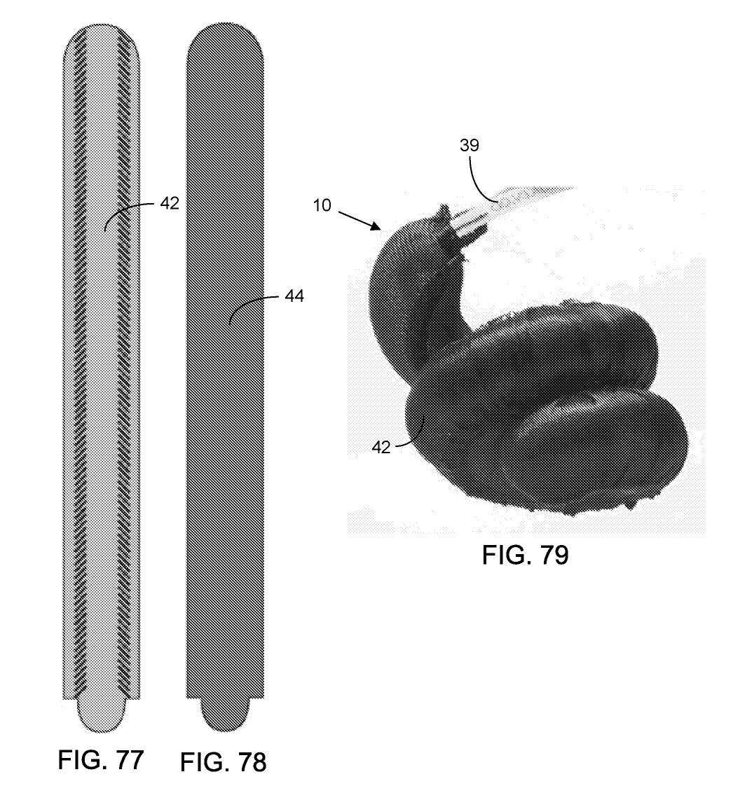

Top and bottom views of a full-gathered actuator 10 with a Merrow seam is shown in FIGS. 77 and 78, respectively. The first fabric layer 42 is a high-stretch gathered knit and is seen in FIG. 77, while the second material layer 44 is a low-stretch knit and is seen in FIG. 78. As seen in FIG. 79, the actuator 10 wraps into a coil when actuated via pressurization.

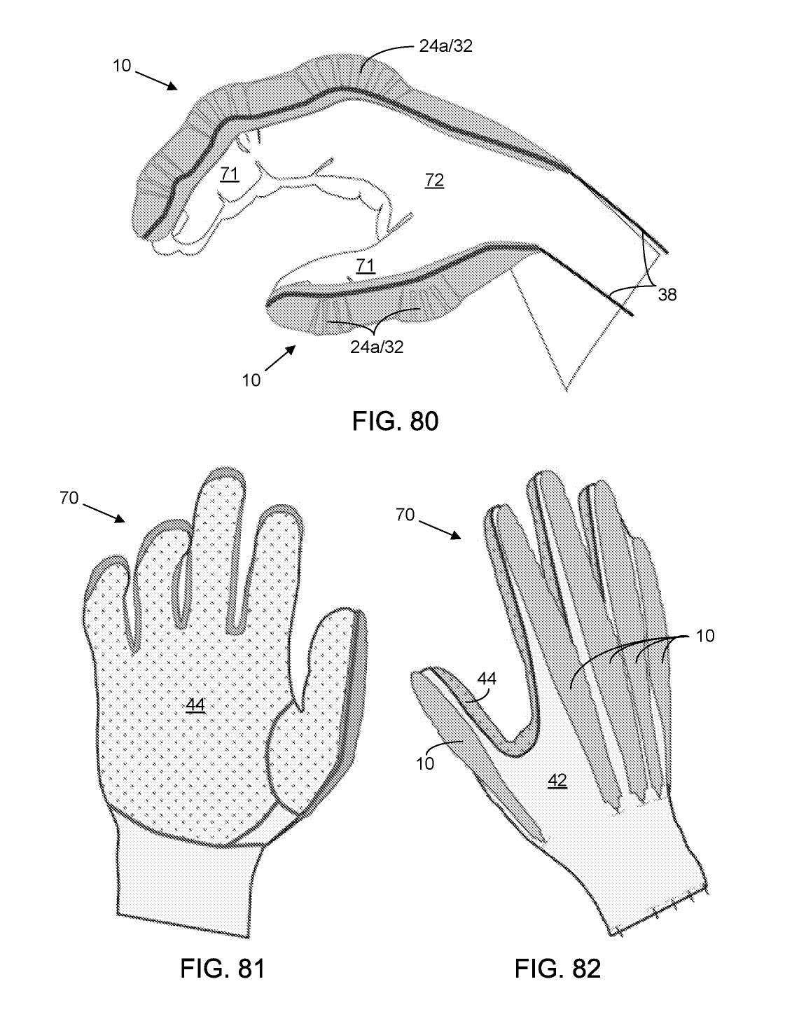

An articulated actuator glove 70 including actuators 10, as disclosed herein, is shown on a human hand 72 in FIGS. 80-82. The first fabric layer 42 and second material layer 44 are shown in FIGS. 81 and 82 but omitted from FIG. 80 to show the positioning of the actuators 10 (particularly the high-stretch knit sections 24a) in relation to the fingers 71 of the hand 72.

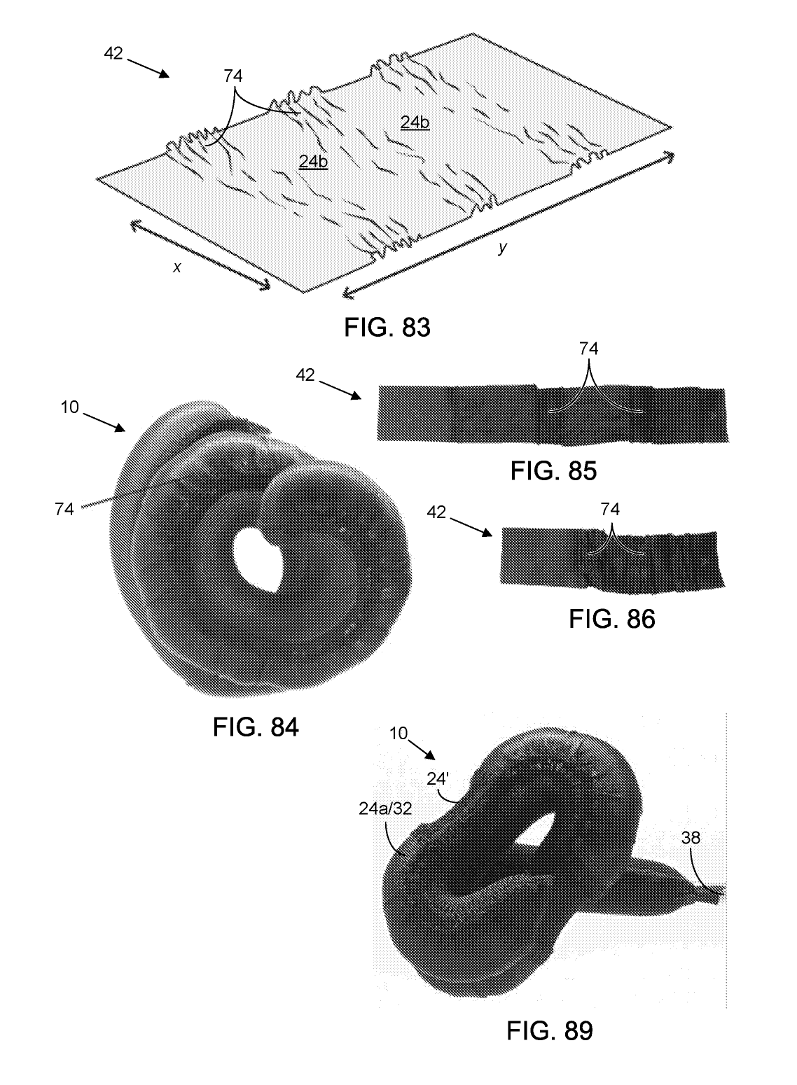

A gathered actuator, created similar to the pleating process where instead of folding the material onto itself in consecutive folds the material is scrunched tightly and sewn at the gathered edge 10 with a Merrow seam is shown in FIG. 83, and a photographic image of this actuator 10 actuated via pressurization is provided in FIG. 84, wherein the wales extend along the x axis, while the courses extend along the y axis. In this embodiment, the gathered material 74 is aligned perpendicular to the grain; and the gathers 73 can arranged along the entire fabric layer or in segments, as shown in FIG. 83. In the illustrated embodiment, the gathers 74 are separated by low-stretch (or no-stretch) fabric sections 24b. When actuated via pressurization, the actuator 10 curls into a coiled structure, as shown in FIG. 84. Photographic images of a first fabric layer 42 with gathered segments 74 are also provided in FIGS. 85 and 86.

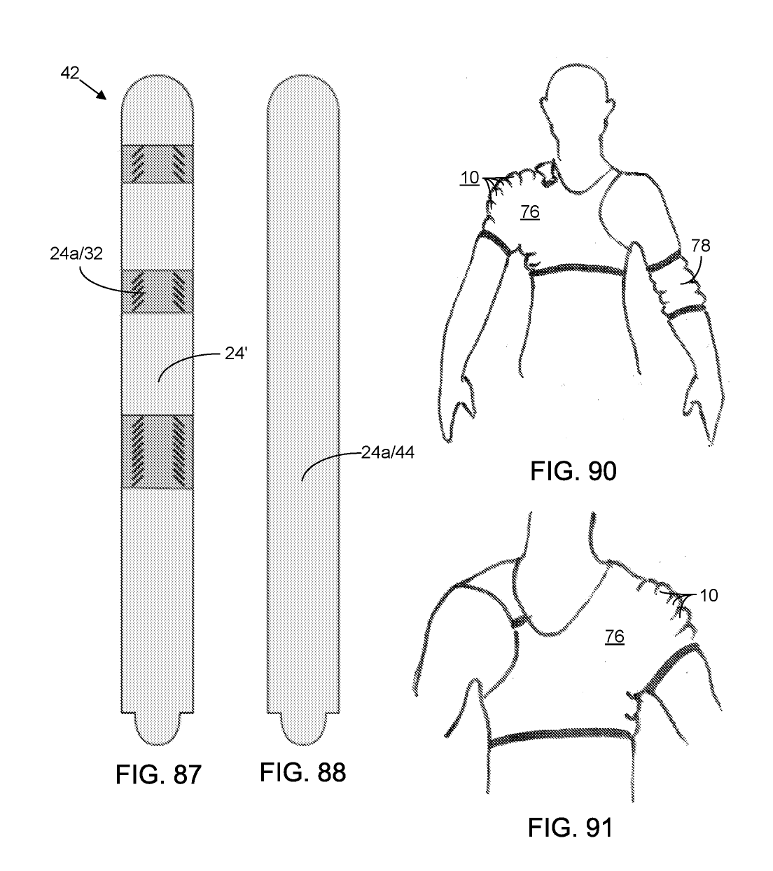

A segmented gathered actuator 10 with a Merrow seam is shown in FIGS. 87-89. The first fabric layer 42, here with sequential segments of a one-way stretch knit 24′ and a gathered high-stretch knit 24a/32, is shown in FIG. 87. The second material layer 44, which is formed of the one-way stretch knit 24′ is shown in FIG. 88. A photographic image of this actuator 10 under pressure actuation is shown in FIG. 89.

In accord with another embodiment, fabric-based fluidic actuators 10 can be designed to only extend upon pressurization. FIG. 32 depicts one construction wherein the first fabric layer 42 and the second material layer 44 mirror each other. The directions of minimal stretch are longitudinally aligned, and the fabric/material layers 42 and 44 are joined by a bond or a seam that permits stretch. Upon fluid pressurization, the actuator 10 will extend in length, as shown in FIG. 33.

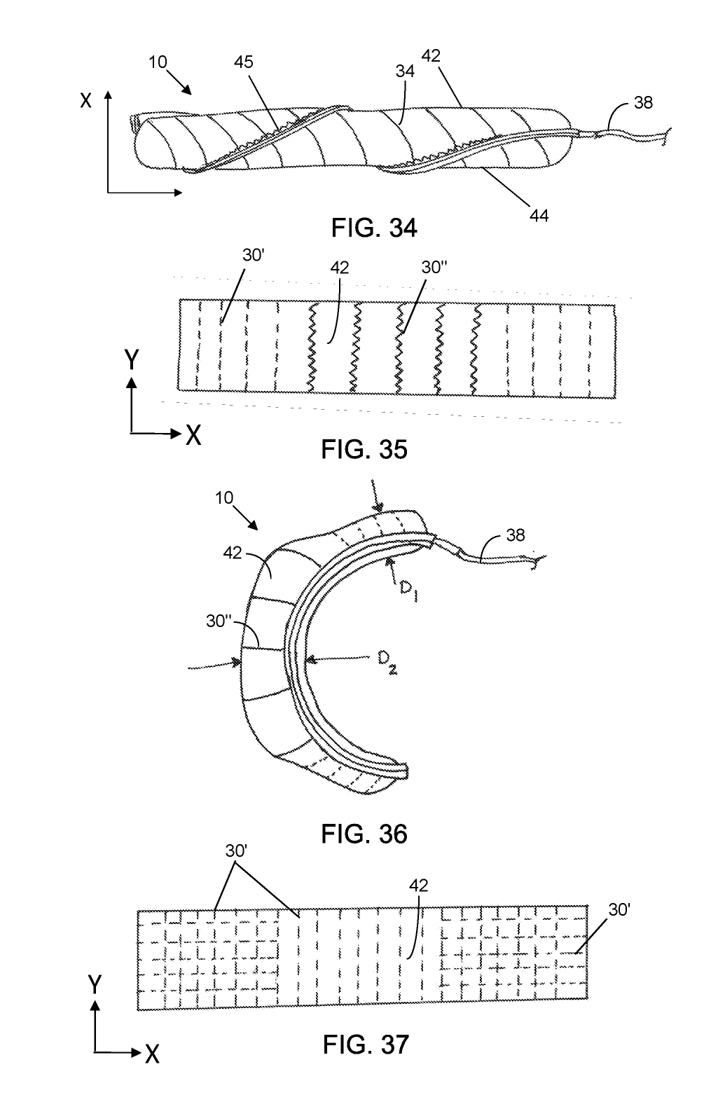

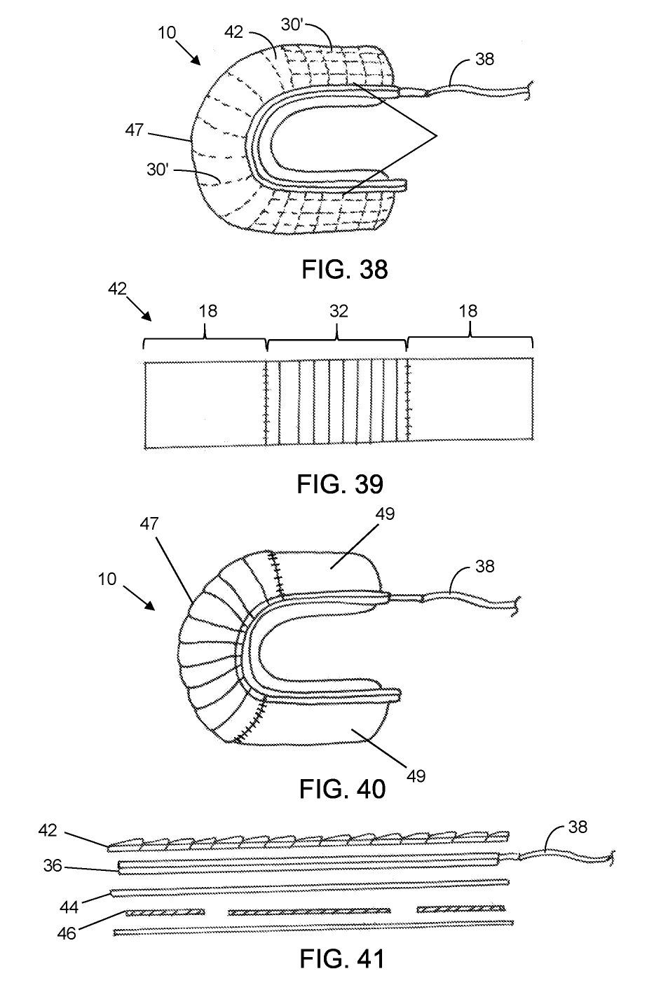

In accord with an additional embodiment, fabric-based fluidic actuators 10 can be designed to twist and extend upon pressurization. In this embodiment, the first and second fabric/material layers 42 and 44 angle the direction of minimal stretch with respect to the longitudinal axis (i.e., in the X-direction), and the fabric/material layers 42 and 44 are joined by a bond or a seam created with a zig-zag stitch that permits stretch. Upon fluid pressurization, the actuator 10 will simultaneously twist and extend in length, as shown in FIG. 34.

In another embodiment, the anisotropic properties of the fabric/material layers 42 and 44 can be varied to alter the resulting shape and/or range of motion of the actuator 10. In one example, for a bending actuator 10, the first fabric layer 42 may have properties that allow varying amounts of stretch circumferentially (i.e., in the Y-direction). FIG. 35 presents a first fabric layer 42 with straight stitch reinforcements 30′ at each end with a section of zig-zag stitch reinforcements 30″ in the middle. The stitch reinforcements 30 do not impede longitudinal stretch (in the X-direction). When this first fabric layer 42 is joined to a second material layer (e.g., in the form of a weave), the actuator's section with zig-zag stitch reinforcements 30″ will swell to a larger diameter, D₂, than the sections with straight stitch reinforcements 30′, D₁, upon pressurization, as shown in FIG. 36. Varying the actuator diameter may be desirable when certain actuator stiffnesses and torques are desired at specific location. This situation may arise if the actuator 10 needs to bend a joint and if minimal bending force is required along a link.

In another specific embodiment, stitch reinforcements 30 can be used to restrict stretch of the fabric/material layer 42/44 in both the X- and Y-directions. FIG. 37 presents a first fabric layer 42 with straight stitch reinforcements 30′ at each end that run in both the X- and Y-directions. A middle portion contains straight stitch reinforcements 30′ that run only in the Y-direction and that permit material stretch in the X-direction. When this first fabric layer 42 is joined to a second material layer 42, such as a woven fabric 18, and the actuator 10 is pressurized (as shown in FIG. 28), the middle portion will bend while the stitch-reinforced end portions will remain relatively straight and will rigidize with increasing pressure.

In another specific embodiment, the first material layer 42 can be assembled with multiple materials (e.g., woven, non-woven and knit materials) to program actuator motions. FIGS. 39 and 40 present an actuator 10 with a first fabric layer 42 where two ends are composed of woven materials 18 that are joined (e.g., bonded or stitched) to a pleated segment 32. When this first fabric layer 42 is combined with an inextensible second material layer 44 to form an actuator 10, the ends of the actuator 10 may be designed to stiffen with increasing pressure while the middle section bends. This multi-segment approach can combine multiple actuator motion types (e.g., bend, bend-twist, extend, extend-twist, rigidizing, etc.) in series or in parallel.

In an alternative embodiment, the first fabric layer 42 is a unitary fabric with a series of patterned knit structures across the length of the fabric, enabling different mechanical functions of the knit in particular zones. Where the fabric is machine-knit, the machine can be programmed to change the structure of the knit as the machine reaches different sections of the fabric being knit. For example, where the actuator is incorporated into a glove, sections that cover joints of the finger can have a more-stretchable knit than other sections of the fabric.

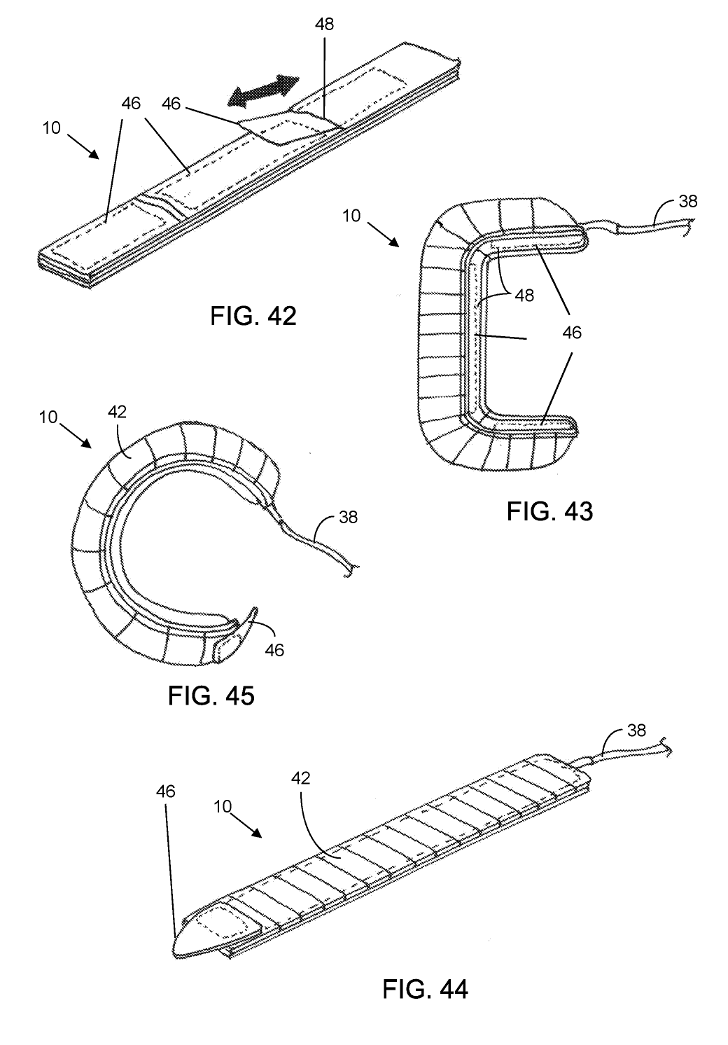

In another embodiment, stiff or rigid inclusions 46 can be integrated into an actuator 10 to restrict motion in a specific zone and to promote motion in others. For rigidizing bladder sections, a thicker material is advantageous (e.g., ˜0.4 mm or thicker). FIG. 41 presents an exploded side view of an actuator assembly 10 where multiple stiff inclusions 46 (i.e., inclusions that are substantially stiffer than the fabric/material layers 42 and 44) are integrated into the actuator 10. These stiff inclusions 46 may be attached by numerous methods including by being sewn, hook-and-loop attached, laced, glued, or heat bonded onto a fabric/material layer 42/44. FIG. 42 presents one embodiment where pockets 48 are created in a third layer 68 (e.g., also in the form of a knit fabric) between the first and second fabric/material layers 42 and 44 enabling the stiff inclusions 46 to be selectively added or removed. Stiff inclusions 46 applied to a bending actuator 10, as shown in FIG. 43, can create joint-like bending where portions of the actuator 10 within the footprint of the stiff inclusion 46 may be restricted from bending, whereas the portions of the fabric/material layers 42 and 44 longitudinally between the stiff inclusions 46 are permitted to bend.

In another embodiment, stiff or rigid inclusions 46 can be attached to the exterior surface of the actuator 10 to augment the physical capabilities of the actuator 10. In one specific embodiment, a stiff inclusion 46 can be added to an actuator 10 to act as a finger nail or finger cap to concentrate forces or to create leverage to lift an object (especially an object that is low in profile, such as a sheet of paper or credit card). Exterior stiff inclusions 46 can also serve as anchor points for attaching actuators 10 to tools or instruments. Stiff inclusions 46 on the exterior surface of one or more of the material/fabric layers can also be used to improve an actuator's abrasion resistance and its resistance to puncture.

In another embodiment, interior and exterior stiff inclusions 46 enable incorporation of electrical and sensing capabilities. Stiff inclusions 46 may take the form of a sensor, a circuit board or a battery. These inclusions 46 may be designed for detecting any or a combination of pressure, force, motion, altitude; and any combination of sensor, circuit board and power can be combined to meet the needs of a specific application.

Stiffening of the actuator 10 can also be achieved via “layer jamming”, wherein at least two layers that can normally slide relative to one another are provided in a pocket 48 of the soft actuator 10. When a vacuum is applied to the pocket 48, the layers are suctioned together, which increases resistance to sliding relative to one another, thereby providing stiffening of the actuator 10. This stiffening can either be along the entire length of the actuator 10 or along just a portion of the actuator 10.

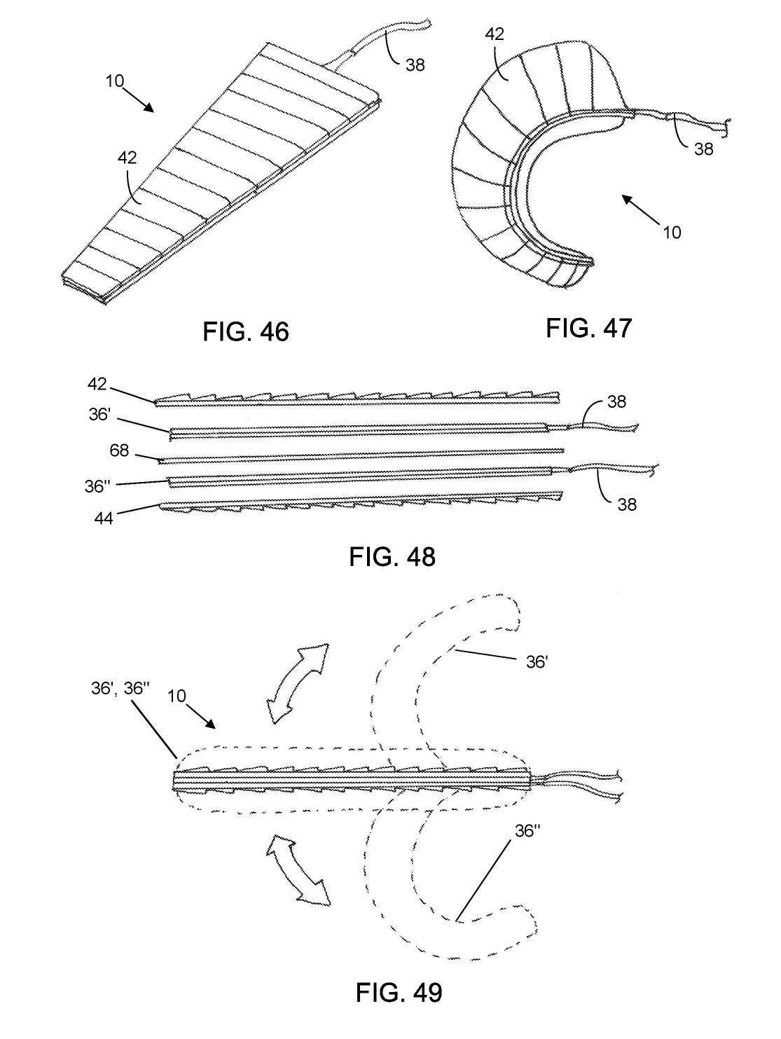

In another embodiment, fabric-based actuators 10 can be fabricated into a range of shapes and geometries. FIG. 46 presents a simple example where the actuator 10 has a tapered shape. If assembled to form a bending actuator 10, this design would result in an actuator 10 that is larger in diameter at one end and narrower at the other end, as shown in FIG. 47. The mechanical properties of this design also produce an actuator 10 that is stiffer and that produces larger forces at one end region (i.e., the larger diameter results in a larger second moment of area that is proportional to stiffness) and that is less stiff and that produces lower forces at the opposite end region.

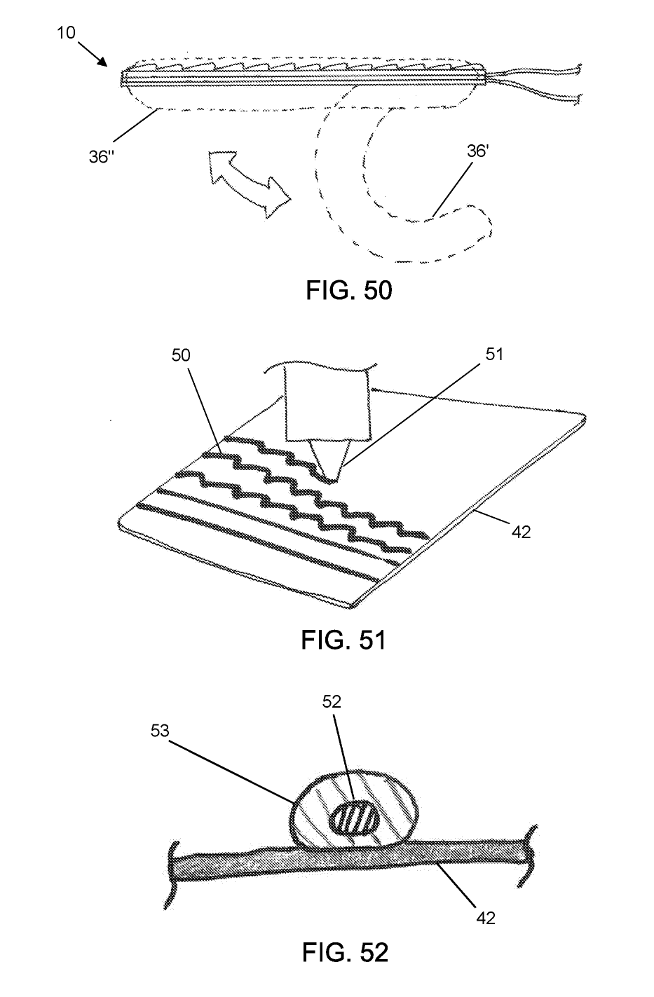

In another embodiment, multiple fabric/material layers 42 and 44 and bladders 36 can be combined to create a variety of actuator geometries and ranges of motion. FIG. 48 presents an exploded side view of components for making a bimorph bending actuator 10. In this specific embodiment, the first fabric layer 42 and third material layer 68 are anisotropic such that they have minimal resistance to stretch along the longitudinal direction and have higher resistance to stretch in the orthogonal or circumferential direction. The second material layer 44 has high strain-limiting properties in both the longitudinal and circumferential directions. In the assembled actuator 10, as shown in FIG. 49, a first bladder 36′ is placed between the first and second fabric/material layers 42/44, and a second bladder 36″ is placed between the second and third material layers 44 and 68. When the first bladder 36′ is pressurized, the fabric-based actuator 10 will curl downward, as depicted by the dotted outline in FIG. 49. When the second bladder 36″ is pressurized (assuming the first bladder 36′ has been evacuated or vents its contents), the actuator 10 will curl upward, as shown in FIG. 49. When both bladders 36 are inflated at the same time, the actuator 10 will remain straight, and its stiffness will increase with pressure, as shown in FIG. 49. Furthermore, varying the timing of inflation of the bladders 36 can be used as a method to influence the radius of curvature of the bending actuator 10 and the resulting stiffness of the actuator 10. In another embodiment, the third material layer 68 can be the same as the second material layer 44 (i.e., possessing high strain-limiting properties), such that, upon pressurization of the second bladder 36″, the actuator 10 straightens to a stiff beam, and pressurization of the first bladder 36′ supports bending, as shown in FIG. 50.

In another embodiment, a bladder 36 can be inserted to influence the range of motion of the actuator 10 while not engaging the anisotropic properties of the fabric/material layers 42 and 44. For example, a bladder 36 with an inflated volume that does not exceed (or that only minimally exceeds) the volume defined between fabric/material layers 42 and 44, and thus does not strain (or minimally strains) the fabric/material layers 42 and 44 and will not generate motions prescribed by the fabric/material layers 42 and 44. Instead, the bladder 36 will rigidize with increasing pressure. In a specific embodiment, the actuator 10 in FIG. 50 can be fabricated with two fabric/material layers 42 and 44 and two bladders 36. The two layers include an anisotropic first fabric layer 42 and a strain-limiting second material layer 44. The two bladders include an oversized first bladder 36′ and a smaller rigidizing second bladder 36″, and the bladders 36 are positioned between the first fabric layer 42 and the second material layer 44. Pressurization of the first bladder 36′ produces the motion prescribed by the fabric/material layers 42 and 44. Pressurization of the second bladder 36″ produces the motion, which depends on the starting configuration of the actuator 10, and shape prescribed the second bladder's rigidizing shape. Furthermore, in some circumstances, it may be desirable to reduce the coefficient of friction between the rigidizing bladder 36 and the fabric/material layers 42 and 44 such that motion of the rigidizing bladder 36 is minimally restricted by the fabric/material layers 42 and 44. Reducing the coefficient of friction can be achieved by several means including selecting low-friction materials for the rigidizing bladder 36, such as polytetrafluoroethylene (PTFE) based plastics or coating or lining the bladder 36 and/or the fabric/materials layers 42 and 44 with a low-friction material.

In another embodiment, a fabric layer's anisotropic properties can be altered by adhering or infusing materials with strain-limiting properties to the first fabric layer 42. This adhesion or infusion can be achieved by any of several methods. In one example, a strain-limiting material 50 can be printed onto the first fabric layer 42 via a print head 51, as shown in FIG. 51. In this example, the strain-limiting material 50 can be a rubber, such as silicone, a thermoplastic composition (e.g., TPU or TPE), or a polyurethane. Direct printing of the strain-limiting material 50 enables rapid customization of the first fabric layer 42 and the ability to modify the texture of the first fabric layer 42. Further, the strain-limiting material 50 can have enhanced properties. For example, fiber reinforcements 52 can be co-extruded with a polymeric (e.g., rubber) material shell 53 to produce a composite strain-limiting material 50 with significantly increased strain-limiting properties, as shown in the cross-section depicted in FIG. 52. Furthermore, the sensors can be co-extruded with the rubber material such that, upon stretch, they produce a measurable change in resistance or capacitance. This sensor can be used to measure the motion of the actuator 10 as it is operated at different pressures or to measure external forces acting on the actuator 10, such as contact with external objects. In another example, the core 52 may be conductive and used in a method to route power or to send and receive signals in a way that can be used to reinforce the first fabric layer 42 or to minimally impede the actuator's range of motion. Furthermore, such sensing cores, conductive cores, and fiber-reinforced cores can be applied together or separately.

In another embodiment, sensors can be added to provide feedback information, such as position/motion of the actuator 10 and the location and magnitude of contact forces. The sensors can take on many forms including soft sensors that consist of elastomeric shells with embedded channels of conductive material that change resistance or capacitance in response to a mechanical deformation, such as strain or pressure. Other sensors can be constructed with electroactive materials, such as electro-static materials or dielectric elastomers. Sensors can also have a fabric construction, such as conductive fabric, where material strain or pressure produces a change in the material's electrical resistance. In one specific example, a first fabric layer 42 can be composed of electrically conductive fabric such that the material layer serves mechanical and sensory roles. A wide variety of conductive fabrics are commercially available; or, alternatively, fabrics can be plated or coated with conductive materials, such as silver, as part of the manufacturing process. Such a technique enables strain or pressure in the fabric to be estimated by measuring a change in resistance. Other parts can be made conductive with metal strands woven into or embroidered onto the construction of the textile or by impregnating textiles components with carbon- or metal-based powders. Furthermore, the sensor may be positioned between fabric/material layers 42 and 44, sewn or infused into a fabric/material layer 42/44, or bonded or mechanically attached to the surface of a fabric/material layer 42/44. In one specific example, a flex sensor, such as a flex sensor manufactured by Spectra Symbol (Salt Lake City, Utah, US), can be placed between or on fabric/material layers 42/44; and the deflection of a bending actuator 10 can be detected by a change in resistance of the flex sensor. In addition to measuring strain and pressure, motion can also be measured by embedding any of a wide variety of sensors (e.g., inertial measurement units, hall-effect sensors, optical sensors) into the fabric actuators as part of the fabrication process. Such a sensor can be secured with fabric or other soft material, glue, or sewing.

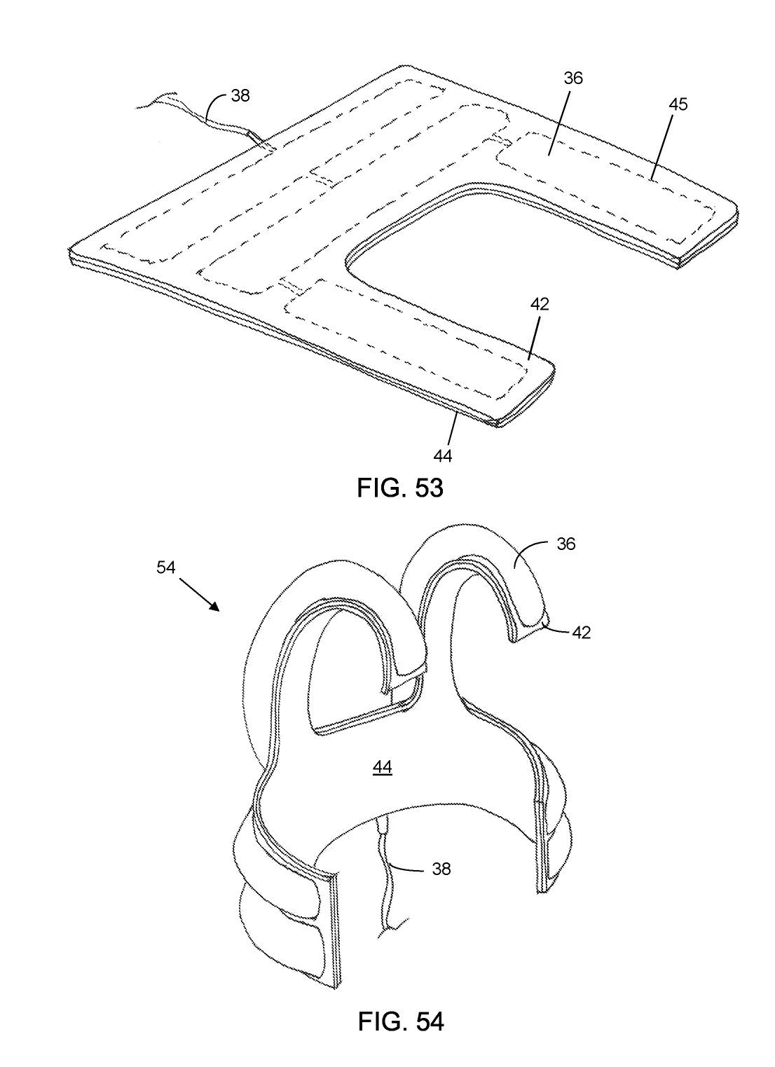

In another embodiment, actuators 10 can be combined in a multitude of configurations. Within two fabric/material layers 42 and 44, multiple actuator types can be defined in different positions and orientations relative to one another to generate a variety of in-plane and out-of-plane motions. This type of configuration is herein referred to as an actuator sheet. Furthermore, multiple actuators 10 in a device can be designed to inflate at the same time or selectively. Selective activation enables portions of the device to remain flexible while others are engaged. FIG. 53 presents an example where two parallel bending actuators 10 run orthogonal to another pair of parallel bending actuators 10. In one application, this structure can be configured into a garment 54 (e.g., a vest that conforms to a wearer's torso and shoulders) when pressurized, as shown in FIG. 54.

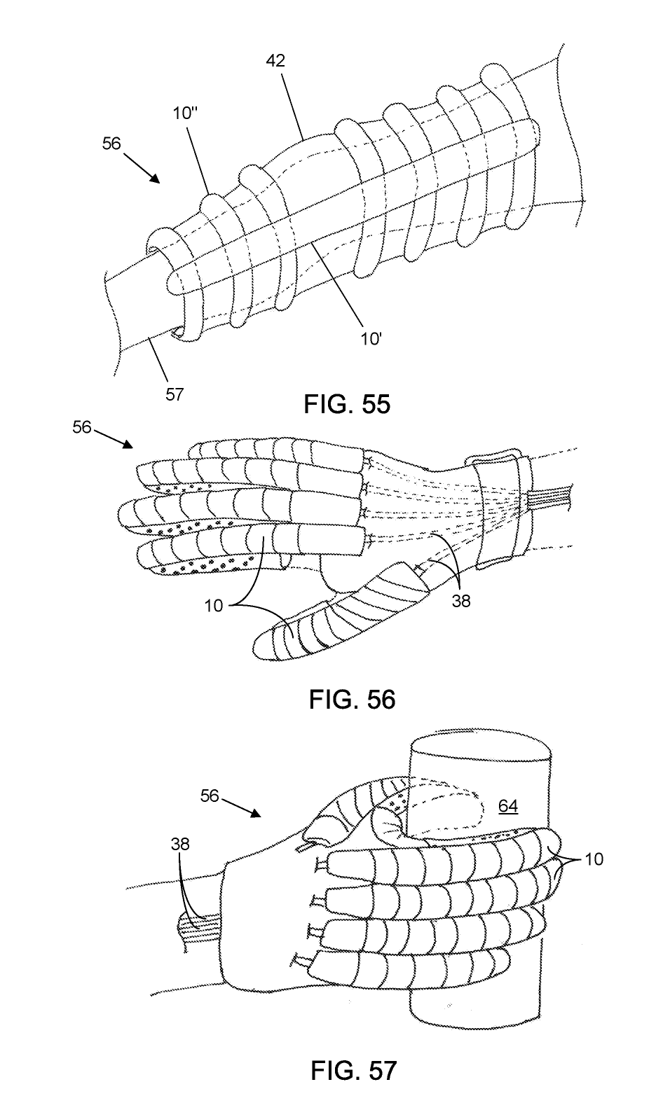

The concept can be extended across multiple fabric/material layers, where multiple actuator types can be configured and layered between multiple fabric/material layers. This methodology can allow more actuators 10 to be packed into the same area and to increase the complexity of the ranges of motions of an actuator sheet. FIG. 55 presents a specific example of this configuration in the form of a wearable device (e.g., brace) 56 for a human leg 57. When all of the actuators 10 are inflated, the device acts as a splint, where a long inflated rigidizing beam actuator 10′ restricts knee bending, and several parallel bending actuators 10″ conform to the wearer's leg 57. The rigidizing beam actuator 10′ and bending actuators 10″ are contained in the same actuator sheet but are positioned between different fabric/material layers 42 and 44. This configuration allows the bending actuators 10″ to seemingly intersect with the rigidizing beam actuator 10′ while still being separate. This configuration can also serve a therapeutic role, where bending actuators 10″ can be selectively inflated in series to create a massaging effect. Furthermore, this configuration can be integrated into clothing where the bending actuators 10″ can be selectively engaged to serve as a tourniquet for severely injured limbs.

In another embodiment, the fabric-based actuators 10 can be configured to support the range of motion of joints (e.g., in hand) of an animal or human. In one specific embodiment, FIG. 56 presents a soft-actuated glove 62 with an actuator 10 for each finger. that the glove 62, when pressurized, can support the hand in closing around an object 64, as shown in FIG. 57. A variety of actuator combinations can be selected depending on the hand pathology. In one scenario, the glove 62 can replace the fingers of an individual with fully or partially amputated hands. In another scenario, the glove 62 can be designed to support hand opening only, a common challenge for stroke survivors with spastic hand(s). In another scenario, the glove 62 can be designed to support hand opening and hand closing for users that have little to no hand strength, as is the case for people suffering from muscular dystrophy or from a spinal cord injury. Furthermore, it should be noted that the above construction methods enable an actuator 10 to be customized to the biomechanics of finger joints such that segments of the actuator 10 bend (or bend-extend) to support joints while other segments (such as those in parallel with bones) may only rigidize. This approach can be further extended to support movement of all joints of the body.

The actuators 10 in the glove 62 (and in other embodiments described herein) can be modular, where, upon failure, an actuator 10 may be removed and replaced with a new actuator 10 without replacing the entire glove 62. This modularity also enables glove customization where actuators 10 can be customized to align with and specifically accommodate each of the fingers such that some actuators 10 may have different geometries, materials, and ranges of motion from adjacent actuators 10 (for example, an actuator for a thumb can designed to execute motions that differ from those of an actuator for one of the other fingers). Alternatively, the actuators 10 may not be modular; but, instead, one can use manufacturing methods similar to those used to create a full glove to create an actuated glove in a few steps, where multiple material layers, pockets, and multiple bladders can be sewn or bonded together.

Those skilled in the art will also appreciate that these fabric-based actuators 10 can be integrated into robotic systems. The versatility of the actuators enables them to support structural roles (i.e., load-bearing rigidizing features) as well as to create motion. In addition to wearable devices, these fabric-based actuators 10 can be designed to make grippers and arms for manipulation and legs for locomotion.

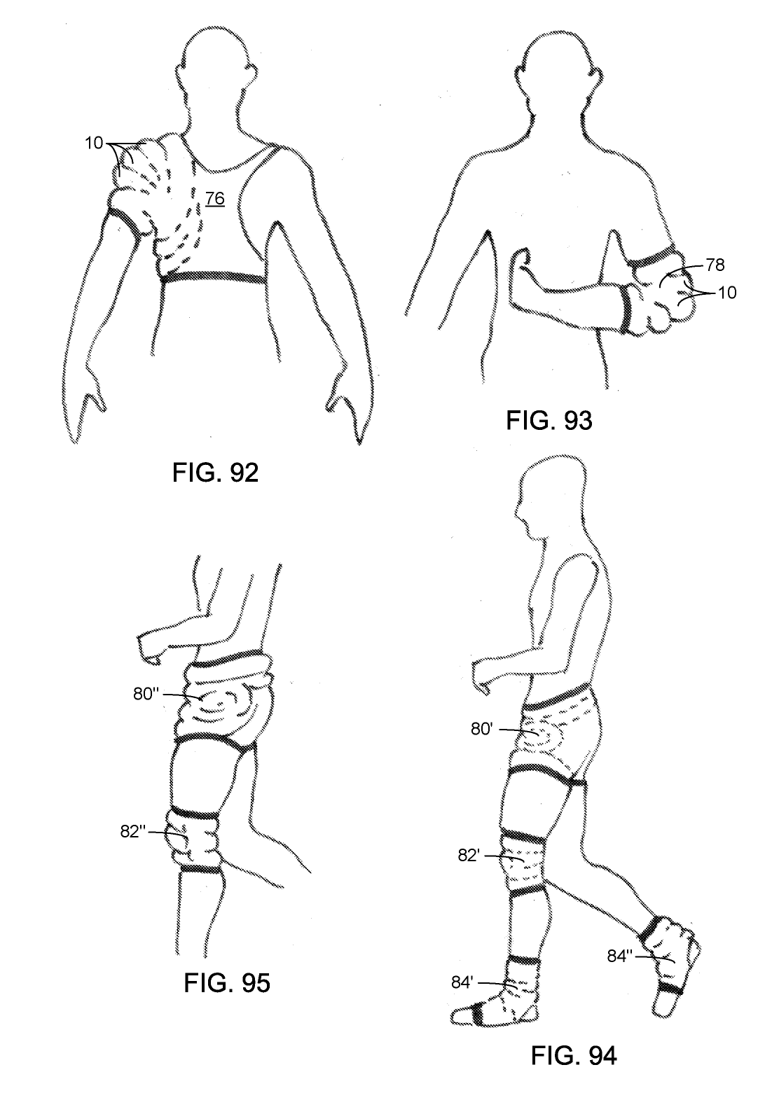

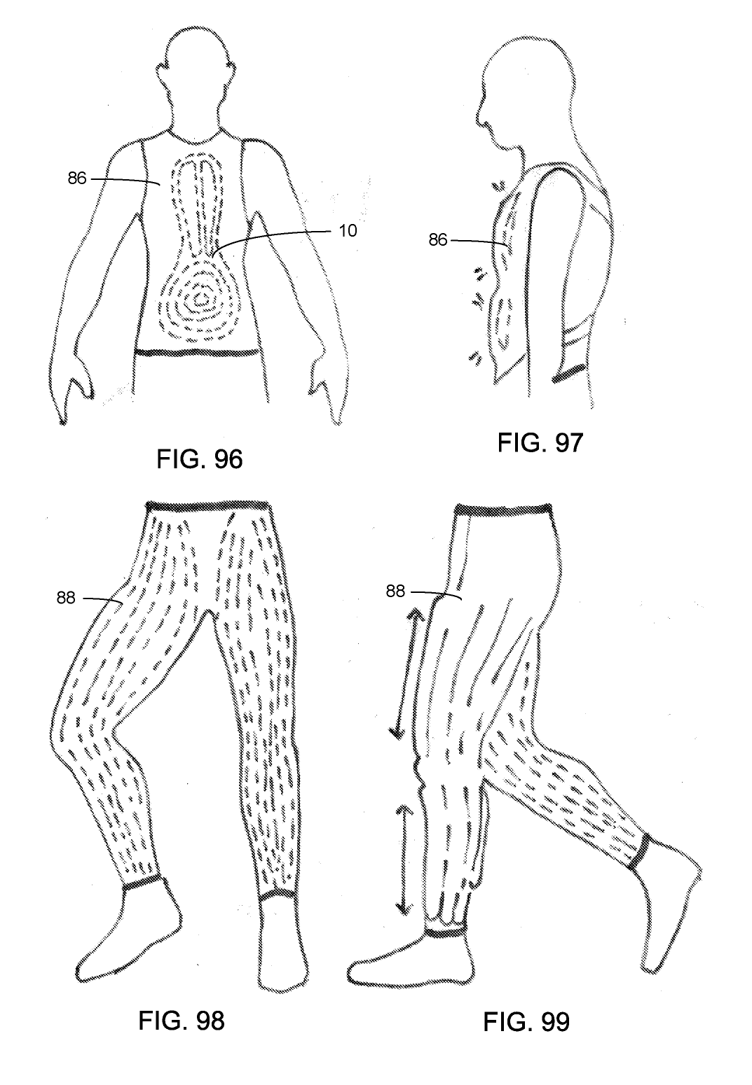

Additionally, the fabric-based actuators can be configured and worn (on a human body) to assist joint movement of, e.g., the shoulder (via a shoulder support 76, as shown in FIGS. 90-92), elbow (via an elbow support 78, as shown in FIGS. 90 and 93), wrist, fingers, hip [via a hip support 80′ (deflated) and 80″ (inflated), as shown in FIGS. 94 and 95], knee [via a knee support 82′ (deflated) and 82″ (inflated), as shown in FIGS. 94 and 95], or ankle [via an ankle support 84′ (deflated) and 84″ (inflated), as shown in FIG. 94]. Further still, the soft-actuators can worn on the upper body (e.g., torso) of a human and, when deflated, be very flexible and non-restrictive, while stiffening and providing support when inflated. In another embodiment, the actuator(s) 10 can be incorporated in a vest 86 worn on a human torso and configured to provide a contracting and expanding displacement to assist breathing, as shown in FIGS. 96 and 97. Full-leg support is provided by the pants 88 incorporating stiffening actuators 10, as shown in FIGS. 98 and 99.

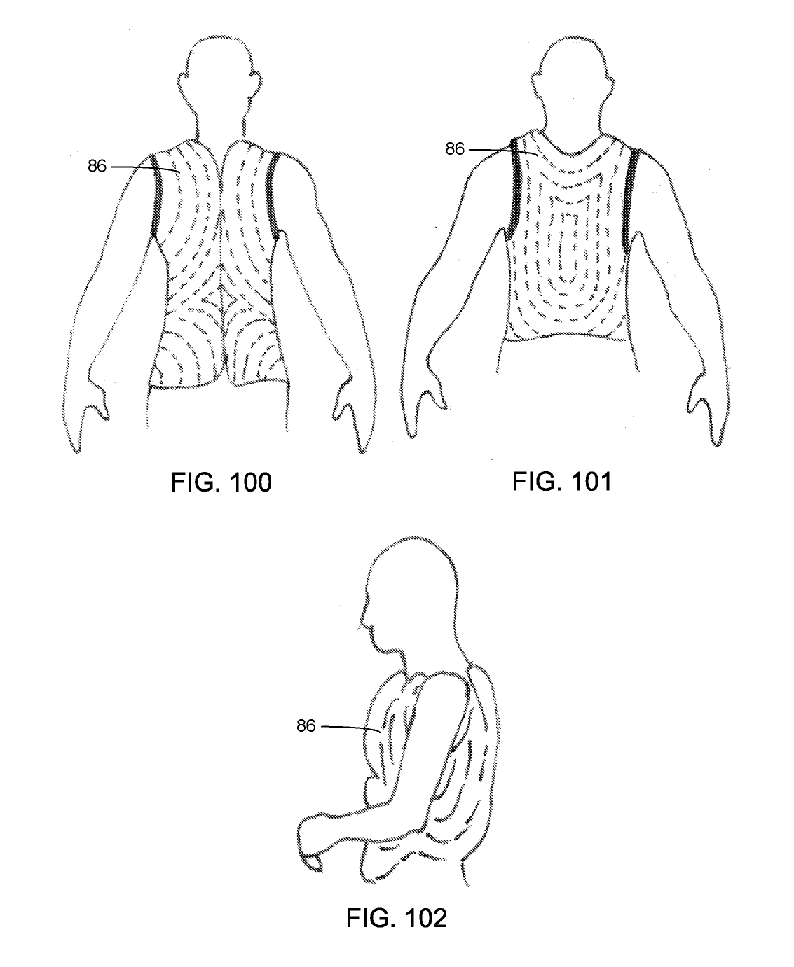

In one embodiment, the actuator(s) can be incorporated in a vehicle (e.g., car, jeep, or truck) safety harness that normally is free and does not restrict movement of the human passenger who wears it; but when the vehicle is moving over rough terrain, a sensor integrated with the device can detect these displacements and actuate the actuators in the brace to stiffen it. Similarly, the actuator(s) can be incorporated into a vest 86, as shown in FIGS. 100-102, that serves as a brace and that is worn outside of a vehicle, e.g., on a human leg or torso so that if that person jumps from a substantial height, then the actuators can stiffen the brace on the leg before impact upon landing. Further still, the actuator(s) can used in a medical application where the actuator(s) are incorporated in a brace worn around a limb to stiffen and support the limb (e.g., a leg, arm or even head or neck) while a person is transported or while the limb is healing.

Further examples consistent with the present teachings are set out in the following numbered clauses:
1. A fabric-based soft actuator, comprising:
   a first fabric layer characterized as having stretch properties selected from (a) anisotropic stretch properties and (b) isotropic stretch properties;
   a second layer characterized as being at least one of (a) a fabric layer with anisotropic or isotropic stretch properties and (b) a strain-limiting layer;
   a bladder disposed between or integrated with the first fabric layer and the second layer; and
   a pressure source in fluid communication with and configured to inflate the bladder.
2. The fabric-based soft actuator of clause 1, wherein the first fabric layer and the second layer are configured to cause the actuator, when actuated, to perform at least one of the following motions: bending, twisting, extending, contracting, rigidizing and combinations thereof.
3. The fabric-based soft actuator of clause 1 or 2, wherein at least one of the first fabric layer and the second layer are configured to generate a plurality of the motions in sequence in the actuator.
4. The fabric-based soft actuator of any of clauses 1-3, wherein the bladder has a thickness no greater than 1 mm.
5. The fabric-based soft actuator of any of clauses 1-4, wherein the anisotropic stretch properties of the first fabric layer are provided by at least one of the following: stitch reinforcements in the first fabric layer, pleating of the first fabric layer, mechanics of the knit or woven structure, and bonding materials with strain-limiting properties adhered to the first fabric layer.
6. The fabric-based soft actuator of any of clauses 1-5, wherein the anisotropic stretch properties of the first fabric layer govern at least one of the following: the shape, force output, and range of motion of the actuator upon actuation.
7. The fabric-based soft actuator of any of clauses 1-6, wherein a plurality of the bladders are included in the actuator between the first fabric layer and the second layer.
8. The fabric-based soft actuator of clause 7, wherein the bladders are combined in the actuator to activate different regions of the actuator.
9. The fabric-based soft actuator of any of clauses 1-8, wherein the first fabric layer includes a plurality of fabrics with different stretch properties.
10. The fabric-based soft actuator of any of clauses 1-8, wherein the first fabric layer includes a plurality of sections, wherein the first fabric layer has a knit structure that differs in different segments.
10.5 The fabric-based soft actuator of any of clauses 1-10, wherein the first fabric layer includes a plurality of sections, wherein a portion of those sections include pleats or gathers.
11. The fabric-based soft actuator of any of clauses 1-10.5, wherein the first fabric layer, the second layer, and the bladder are configured to provide a plurality of degrees of freedom for actuator motion.
12. The fabric-based soft actuator of any of clauses 1-11, further comprising at least one stiff inclusion that is stiffer than the first fabric layer incorporated in, on or between fabric layers.
13. The fabric-based soft actuator of clause 12, wherein the stiff inclusion provides at least one of the following functions: altering the range of motion of the actuator, providing a mounting or connection point, abrasion resistance, sensing capability, and substrate for a rigid element such as a circuit board, battery, microprocessor, or a light-emitting diode.
14. The fabric-based soft actuator of any of clauses 1-13, wherein the bladder is configured to rigidize the actuator before the first fabric layer stretches.
15. The fabric-based soft actuator of any of clauses 1-14, wherein the actuator is mounted to clothing.
16. The fabric-based soft actuator of any of clauses 1-15, wherein the actuator is worn by an organism.
17. The fabric-based soft actuator of any of clauses 1-16, wherein the organism is a human.
18. The fabric-based soft actuator of any of clauses 1-17, wherein the actuator supports at least one joint motion of the human.
19. The fabric-based soft actuator of any of clauses 1-18, wherein the actuator restricts at least one direction of motion at a joint of the human to reduce a risk of damage to the joint.
20. The fabric-based soft actuator of any of clauses 1-19, wherein the bladder includes a rigidizing bladder having a coefficient of friction below 0.3.
21. The fabric-based soft actuator of any of clauses 1-20, further comprising an electrically conducting material integrated into or added to the first fabric layer.
22. The fabric-based soft actuator of any of clauses 1-21, further comprising a strain sensor integrated into or added to the first fabric layer, wherein the strain sensor is selected from conductive thread and soft sensors, wherein the strain sensor changes resistance or capacitance with strain to detect strain of the first fabric layer.
23. The fabric-based soft actuator of any of clauses 1-22, further comprising a motion sensor integrated into or added to the first fabric layer, wherein the motion sensor is selected from inertial measurement units, flex sensors, hall-effect sensors, and optical sensors, wherein the motion sensor is configured to detect motion of the actuator.
24. A gripper, comprising a plurality of the actuators of any of clauses 1-24 configured to grab objects.
25. A method for actuation utilizing the fabric-based soft actuator of any of clauses 1-23, the method comprising pumping fluid into or out of the bladder to displace or stiffen the fabric-based soft actuator.
26. The method of clause 26, wherein the fabric-based actuator is worn on at least a portion of a body of an organism (e.g., a human), and wherein the displacement or stiffening of the fabric-based soft actuator assists or restricts movement or acts as a brace against the body.

In describing embodiments of the invention, specific terminology is used for the sake of clarity. For the purpose of description, specific terms are intended to at least include technical and functional equivalents that operate in a similar manner to accomplish a similar result. Additionally, in some instances where a particular embodiment of the invention includes a plurality of system elements or method steps, those elements or steps may be replaced with a single element or step. Likewise, a single element or step may be replaced with a plurality of elements or steps that serve the same purpose. Further, where parameters for various properties or other values are specified herein for embodiments of the invention, those parameters or values can be adjusted up or down by 1/100^{th}, 1/50^{th}, 1/20^{th}, 1/10^{th}, ⅕^{th}, ⅓^{rd}, ½, ⅔^{rd}, ¾^{th}, ⅘^{th}, 9/10^{th}, 19/20^{th}, 49/50^{th}, 99/100^{th}, etc. (or up by a factor of 1, 2, 3, 4, 5, 6, 8, 10, 20, 50, 100, etc.), or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention. Further still, other aspects, functions, and advantages are also within the scope of the invention; and all embodiments of the invention need not necessarily achieve all of the advantages or possess all of the characteristics described above. Additionally, steps, elements and features discussed herein in connection with one embodiment can likewise be used in conjunction with other embodiments. The contents of references, including reference texts, journal articles, patents, patent applications, etc., cited throughout the text are hereby incorporated by reference in their entirety; and appropriate components, steps, and characterizations from these references may or may not be included in embodiments of this invention. Still further, the components and steps identified in the Background section are integral to this disclosure and can be used in conjunction with or substituted for components and steps described elsewhere in the disclosure within the scope of the invention. In method claims (or where methods are elsewhere recited), where stages are recited in a particular order—with or without sequenced prefacing characters added for ease of reference—the stages are not to be interpreted as being temporally limited to the order in which they are recited unless otherwise specified or implied by the terms and phrasing.

1. A fabric-based soft actuator, comprising:
a first fabric layer characterized as having stretch properties selected from (a) anisotropic stretch properties and (b) isotropic stretch properties;
a second layer characterized as being at least one of (a) a fabric layer with anisotropic or isotropic stretch properties and (b) a strain-limiting layer;
a bladder disposed between or integrated with the first fabric layer and the second layer; and
a pressure source in fluid communication with and configured to inflate the bladder.

2. The fabric-based soft actuator of claim 1, wherein the first fabric layer and the second layer are configured to cause the actuator, when actuated, to perform at least one of the following motions: bending, twisting, extending, contracting and combinations thereof.

3. The fabric-based soft actuator of claim 2, wherein at least one of the first fabric layer and the second layer are configured to generate a plurality of the motions in sequence in the actuator.

4. The fabric-based soft actuator of claim 1, wherein the bladder has a thickness no greater than 1 mm.

5. The fabric-based soft actuator of claim 1, wherein the anisotropic stretch properties of the first fabric layer are provided by at least one of the following: stitch reinforcements in the first fabric layer, pleating, scrunching, or gathering of the first fabric layer, mechanics of the knit or woven structure, bonding materials with strain-limiting properties adhered to the first fabric layer, and reinforcing material printed on the first fabric layer.

6. The fabric-based soft actuator of claim 1, wherein the anisotropic stretch properties of the first fabric layer govern at least one of the following: the shape, force output, and range of motion of the actuator upon actuation.

7. The fabric-based soft actuator of claim 1, wherein a plurality of the bladders are included in the actuator between the first fabric layer and the second layer.

8. The fabric-based soft actuator of claim 7, wherein the bladders are combined in the actuator to activate different regions of the actuator.

9. The fabric-based soft actuator of claim 1, wherein the first fabric layer includes a plurality of fabrics with different stretch properties.

10. The fabric-based soft actuator of claim 1, wherein the first fabric layer includes a plurality of sections, wherein the first fabric layer has a knit structure that differs in different segments.

11. The fabric-based soft actuator of claim 1, wherein the first fabric layer includes a plurality of sections, wherein a portion of those sections include pleats or gathers.

12. The fabric-based soft actuator of claim 1, wherein the first fabric layer, the second layer, and the bladder are configured to provide a plurality of degrees of freedom for actuator motion.

13. The fabric-based soft actuator of claim 1, further comprising at least one stiff inclusion that is stiffer than the first fabric layer incorporated in, on or between fabric layers.

14. The fabric-based soft actuator of claim 13, wherein the stiff inclusion provides at least one of the following functions: altering the range of motion of the actuator, providing a mounting or connection point, abrasion resistance, sensing capability, and substrate for a circuit board, battery, microprocessor, or a light-emitting diode.

15. The fabric-based soft actuator of claim 1, wherein the bladder is configured to rigidize the actuator before the first fabric layer stretches.

16. The fabric-based soft actuator of claim 1, wherein the actuator is mounted to clothing.

17. The fabric-based soft actuator of claim 1, wherein the bladder includes a rigidizing bladder having a coefficient of friction below 0.3.

18. The fabric-based soft actuator of claim 1, further comprising an electrically conducting material integrated into or added to the first fabric layer.

19. The fabric-based soft actuator of claim 1, further comprising a strain sensor integrated into or added to the first fabric layer, wherein the strain sensor is selected from conductive thread and soft sensors, wherein the strain sensor changes resistance or capacitance with strain to detect strain of the first fabric layer.

20. The fabric-based soft actuator of claim 1, further comprising a motion sensor integrated into or added to the first fabric layer, wherein the motion sensor is selected from inertial measurement units, flex sensors, hall-effect sensors, and optical sensors, wherein the motion sensor is configured to detect motion of the actuator.

21. A gripper, comprising a plurality of the actuators of claim 1 configured to grasp objects.

22. A method for actuation utilizing a fabric-based soft actuator comprising a first fabric layer having stretch properties selected from anisotropic stretch properties and isotropic stretch properties, a second layer characterized as being at least one of a fabric layer with anisotropic or isotropic stretch properties and a strain-limiting layer, and a bladder between the first fabric layer and the second layer, the method comprising pumping fluid into or out of the bladder to displace or stiffen the fabric-based soft actuator.

23. The method of claim 22, wherein the fabric-based actuator is worn on at least a portion of a body of an organism, and wherein the displacement or stiffening of the fabric-based soft actuator assists or restricts movement or acts as a brace against the body.

24. The method of claim 23, wherein the organism is a human.
